SEQUENCE LISTING (47 sequences)

SEQ ID NO: 1 - protein, 9 aa, Homo sapiens
FYISPVNKL

SEQ ID NO: 2 - protein, 9 aa, Homo sapiens
HLSDAIVEV

SEQ ID NO: 3 - protein, 13 aa, Homo sapiens
LPFLALPDGAHNY

SEQ ID NO: 4 - protein, 9 aa, Homo sapiens
LYGLLQAKL

SEQ ID NO: 5 - protein, 8 aa, Homo sapiens
YISPVNKL

SEQ ID NO: 6 - DNA, 2353 bp, Homo sapiens
gctttgcctccaccgatctccctgtggggccctcatgtgctgtgctcgctgacacccgaagtccgcggctttccgcacacggtggggtcgtcagacccgctgcccttggcggtcgaagtcgtcgtgcgggcccgcggcggccgcccatggagaaggccaggagaggcggggatggcgtcccccgggggcccgtactgcacatcgtggtggtcggatttcaccacaagaagggctgccaggttgaattctcttacccgcccctgattccaggagatggacatgacagccacactttacctgaagaatggaagtatttgcccttccttgccttaccagatggcgcacacaactaccaggaagatactgtgttttttcacttgccacccagaaatggaaatggagccacagtatttggtatctcttgctatcgacaaattgaagccaaggcactgaaagtaaggcaagcagatatcaccagagagactgttcagaaaagtgtctgtgttctaagcaagctgcctctgtatggtttacttcaagcaaaacttcaactcattacacatgcatattttgaagagaaggatttttcccaaatttctattctaaaggagctttatgaacatatgaatagttccttgggaggtgcttcattagaaggatcccaagtatatcttggtctgtcacctcgagatcttgtccttcattttcgacacaaggtcttaatcctatttaagctaattcttcttgaaaaaaaggttcttttttatatttctccagtgaataaattggtgggtgcactgatgactgtgttatccctttttccaggcatgattgaacatggtctcagtgactgttctcagtatagaccccggaaaagtatgtctgaagatggtgggcttcaggaaagtaacccatgtgcagatgattttgtttctgcatccactgctgatgtttcacataccaacttgggaactatcaggaaagtcatggcaggaaaccatggagaagatgctgccatgaagactgaggagcctttgttccaagtggaagacagcagcaaagggcaggaacccaatgataccaatcaatatttgaaacctccatctcgcccatctccagattcttcagaaagtgactgggaaactttggatcctagtgtcttagaggaccccaacttgaaagaaagggaacagctgggatcagaccagacaaatttgtttccaaaggactctgtcccctcagagagtcttccaattactgtacaacctcaagctaatacgggacaggtagtcctgataccagggctcatttcgggtttggaagaggatcagtatggcatgcccctggccatcttcacaaagggatatctgtgtttgccttacatggcattgcagcagcatcatcttctctccgatgtcaccgttcgggggtttgttgctggagctactaacatcctttttcgacaacagaaacacctcagtgatgccattgtggaagtagaagaagctctgatccagatccatgatccagaactcaggaagctgcttaacccaaccactgcagacctaaggttcgcagactacctagtgaggcacgtgactgagaatcgggatgacgtcttcctagatggcacgggctgggagggaggtgacgaatggatccgggcccagtttgcggtctacattcatgccctgctggctgccacgctgcaattagacaatgaaaagatattatcggactatgggacaacttttgttacagcatggaagaatactcacaactacagggtgtggaacagcaacaagcatccagcacttgcagaaataaatccaaaccatccatttcaaggccaatactcagtatcagacatgaagttaaggttctcacattctgttcagaatagtgaacgtggcaaaaaaattggaaacgtcatggtcacaactagccggaatgttgtacaaacaggaaaagctgttggccagtcagttggaggagctttttccagtgcaaagacagctatgtcttcatggctttccactttcaccacttccacctcccaaagtctcactgagccaccagatgagaagccttgagcaaggcgtcagaggctgctattgctttctgaggtttaagtgtcccctgtctgtctgctgctcccaggctgttactagccacagatccacagcaggggaccatatgtcgaactgtttacatggatgttgctctaagtgaatgtttcgggatgccgaaatgatgaaatcacagccatagcagggatggctttccaggttggggtttcaattgactacttttatttcagtctgagcctgattaaaacatacagtgaaccttc

SEQ ID NO: 7 - protein, 648 aa, Homo sapiens
MEKARRGGDGVPRGPVLHIVVVGFHHKKGCQVEFSYPPLIPGDGHDSHTLPEEWKYLPFLALPDGAHNYQEDTVFFHLPPRNGNGATVFGISCYRQIEAKALKVRQADITRETVQKSVCVLSKLPLYGLLQAKLQLITHAYFEEKDFSQISILKELYEHMNSSLGGASLEGSQVYLGLSPRDLVLHFRHKVLILFKLILLEKKVLFYISPVNKLVGALMTVLSLFPGMIEHGLSDCSQYRPRKSMSEDGGLQESNPCADDFVSASTADVSHTNLGTIRKVMAGNHGEDAAMKTEEPLFQVEDSSKGQEPNDTNQYLKPPSRPSPDSSESDWETLDPSVLEDPNLKEREQLGSDQTNLFPKDSVPSESLPITVQPQANTGQVVLIPGLISGLEEDQYGMPLAIFTKGYLCLPYMALQQHHLLSDVTVRGFVAGATNILFRQQKHLSDAIVEVEEALIQIHDPELRKLLNPTTADLRFADYLVRHVTENRDDVFLDGTGWEGGDEWIRAQFAVYIHALLAATLQLDNEKILSDYGTTFVTAWKNTHNYRVWNSNKHPALAEINPNHPFQGQYSVSDMKLRFSHSVQNSERGKKIGNVMVTTSRNVVQTGKAVGQSVGGAFSSAKTAMSSWLSTFTTSTSQSLTEPPDEKP

SEQ ID NO: 8 - protein, 10 aa, Homo sapiens
LYQILQGIVF

SEQ ID NO: 9 - protein, 9 aa, Homo sapiens
SYNPLWLRI

SEQ ID NO: 10 - protein, 9 aa, Homo sapiens
NYLPFIMEL

SEQ ID NO: 11 - protein, 9 aa, Homo sapiens
SYIDVLPEF

SEQ ID NO: 12 - protein, 9 aa, Homo sapiens
SYIIDPLNL

SEQ ID NO: 13 - protein, 10 aa, Homo sapiens
YYNAAGFNKL

SEQ ID NO: 14 - protein, 9 aa, Homo sapiens
NYLLYVSNF

SEQ ID NO: 15 - protein, 9 aa, Homo sapiens
AYLVYTDRL

SEQ ID NO: 16 - protein, 9 aa, Homo sapiens
HYKPTPLYF

SEQ ID NO: 17 - protein, 10 aa, Homo sapiens
VWSDVTPLTF

SEQ ID NO: 18 - protein, 15 aa, Homo sapiens
TLGEFLKLDRERAKN

SEQ ID NO: 19 - protein, 16 aa, Homo sapiens
DDPSTIEKLAKNKQKP

SEQ ID NO: 20 - protein, 17 aa, Homo sapiens
NKQKPITPETAEKLARD

SEQ ID NO: 21 - protein, 17 aa, Homo sapiens
NGAYKAIPVAQDLNAPS

SEQ ID NO: 22 - protein, 15 aa, Homo sapiens
TLGEFLKLDRERAKD

SEQ ID NO: 23 - protein, 9 aa, Homo sapiens
FTELTLGEF

SEQ ID NO: 24 - protein, 9 aa, Homo sapiens
LMLGEFLKL

SEQ ID NO: 25 - protein, 9 aa, Homo sapiens
EPDLAQCFY

SEQ ID NO: 26 - protein, 9 aa, Homo sapiens
VYGIRLEHF

SEQ ID NO: 27 - protein, 9 aa, Homo sapiens
TYGNLLDYL

SEQ ID NO: 28 - protein, 9 aa, Homo sapiens
RFLSGIINF

SEQ ID NO: 29 - protein, 9 aa, Homo sapiens
VYTTSYQQI

SEQ ID NO: 30 - protein, 9 aa, Homo sapiens
NYEETFPHI

SEQ ID NO: 31 - protein, 9 aa, Homo sapiens
RYLWATVTI

SEQ ID NO: 32 - protein, 9 aa, Homo sapiens
VYFSKSEQL

SEQ ID NO: 33 - protein, 9 aa, Homo sapiens
VFIFKGNQF

SEQ ID NO: 34 - protein, 9 aa, Homo sapiens
QYASRFVQL

SEQ ID NO: 35 - protein, 9 aa, Homo sapiens
KYLTVKDYL

SEQ ID NO: 36 - protein, 9 aa, Homo sapiens
VYNPTPNSL

SEQ ID NO: 37 - protein, 9 aa, Homo sapiens
SYLQAANAL

SEQ ID NO: 38 - protein, 9 aa, Homo sapiens
FYQPKIQQF

SEQ ID NO: 39 - protein, 9 aa, Homo sapiens
YYKNIGLGF

SEQ ID NO: 40 - protein, 9 aa, Homo sapiens
AYAIIKEEL

SEQ ID NO: 41 - protein, 9 aa, Homo sapiens
LYPEVFEKF

SEQ ID NO: 42 - protein, 10 aa, Homo sapiens
KYNDTFWKEF

SEQ ID NO: 43 - protein, 10 aa, Homo sapiens
VFDTAIAHLF

SEQ ID NO: 44 - protein, 9 aa, Homo sapiens
VYPNWAIGL

SEQ ID NO: 45 - protein, 9 aa, Homo sapiens
VYKVVGNLL

SEQ ID NO: 46 - protein, 9 aa, Homo sapiens
VYIEKNDKL

SEQ ID NO: 47 - protein, 10 aa, Homo sapiens
IYNGKLFDLL

(19) United States
(12) Patent Application Publication
(10) Pub. No.: US 2011/0229505 A1
(43) Pub. Date: Sep. 22, 2011

(54) Methods for the diagnosis and treatment of cancer based on AVL9

(75) Inventors: Jens FRITSCHE, Tuebingen (DE); Toni WEINSCHENK, Aichwald (DE); Steffen WALTER, Reutlingen (DE); Peter LEWANDROWSKI, Tuebingen-Hirschau (DE); Harpreet SINGH, Tuebingen (DE); Oliver SCHOOR, Tuebingen (DE)

(73) Assignee: IMMATICS BIOTECHNOLOGIES GMBH, Tuebingen (DE)

(21) Appl. No.: 13/051,701

(22) Filed: Mar. 18, 2011

Related U.S. Application Data
(60) Provisional application No. 61/315,715, filed on Mar. 19, 2010.
(60) Provisional application No. 61/315,704, filed on Mar. 19, 2010.
(60) Provisional application No. 61/414,251, filed on Nov. 16, 2010.

(30) Foreign Application Priority Data
Mar. 19, 2010 (GB) 1004551.6
Mar. 19, 2010 (GB) 1004575.5

(51) Int. Cl. A61K 39/00; C12N 15/63; C12N 5/0783; C12N 5/10; C12N 1/00; C12Q 1/68; G01N 33/574; C07K 14/47; C07K 7/06; C07K 7/08; C07K 19/00; C07H 21/00; A61K 38/17; C07K 16/18; A61K 48/00; A61K 35/00; A61P 35/00; A61P 37/04

(52) U.S. Cl. 424/185.1; 435/320.1; 435/325; 435/419; 435/243; 435/6.14; 435/7.23; 530/350; 435/69.3; 530/328; 530/327; 530/326; 530/325; 530/324; 536/23.5; 514/19.3; 424/277.1; 530/387.9; 514/44 R; 424/93.2; 435/375; 435/6.11

(57) ABSTRACT

The present invention relates to methods for the diagnosis and treatment of cancer in mammals, in particular gastric cancer, based on the new target AVL9. The present invention thus relates to diagnostic methods and related components to be used in such methods. Furthermore, the present invention relates to the treatment of cancer in mammals, in particular gastric cancer, based on AVL9 as a target. Specifically, the present invention relates to the immunotherapy of cancer using AVL9 tumor-associated cytotoxic T cell (CTL) peptide epitopes, alone or in combination with other tumor-associated peptides, and respective pharmaceutical compositions, in particular vaccine compositions.

### RELATED APPLICATIONS

This application claims priority to U.S. Provisional Patent Application 61/315,704, U.S. Provisional Patent Application 61/315,715, UK Patent Application GB1004551.6, and UK Patent Application GB1004575.5, each of which was filed on Mar. 19, 2010, and U.S. Provisional Patent Application 61/414,251, filed on Nov. 16, 2010, the entire contents of which are hereby incorporated by reference.

### FIELD OF THE INVENTION

The present invention relates to methods for the diagnosis and treatment of cancer in mammals, in particular gastric cancer, based on the new target AVL9. The present invention thus relates to diagnostic methods and related components to be used in such methods. Furthermore, the present invention relates to the treatment of cancer in mammals, in particular gastric cancer, based on AVL9 as a target. Specifically, the present invention relates to the immunotherapy of cancer using AVL9 tumor-associated cytotoxic T cell (CTL) peptide epitopes, alone or in combination with other tumor-associated peptides, and respective pharmaceutical compositions, in particular vaccine compositions.

### BACKGROUND OF THE INVENTION

Gastric cancer is a disease in which malignant cells are formed in the lining of the stomach. Stomach or gastric cancer can develop in any part of the stomach and may spread throughout the stomach and to other organs; particularly the esophagus, lungs and the liver. Stomach cancer is the fourth most common cancer worldwide with 930,000 cases diagnosed in 2002. It has a high mortality rate (˜800,000 per year) making it the second most common cause of cancer death worldwide.

Standard treatment for gastric cancer may involve surgery, chemotherapy, radiation therapy or chemoradiation. Surgery is the primary treatment for gastric cancer. The goal of surgery is to accomplish a complete resection with negative margins (R0 resection). However, approximately 50% of patients with locoregional gastric cancer cannot undergo an R0 resection. R1 indicates microscopic residual cancer (positive margins); and R2 indicates gross (macroscopic) residual cancer but not distant disease. Thus, patient outcome heavily depends on the initial stage of the cancer at diagnosis.

Gastric cancer is more common in men, and has a higher frequency in Asian and developing countries. Tremendous geographic variation exists in the incidence of this disease around the world. Rates of the disease are highest in Asia and parts of South America and lowest in North America. The highest death rates are recorded in Chile, Japan, South America, and the former Soviet Union. Gastric cancer is the leading cancer type in Korea, with 20.8% of malignant neoplasms. In Japan, gastric cancer remains the most common cancer for men. Each year in the United States, about 13,000 men and 8,000 women are diagnosed with stomach cancer, thus representing roughly 2% (25,500 cases) of all new cancer cases yearly in the United States. Most patients are older than 70 years.

Gastric cancer is often diagnosed at an advanced stage, because screening is not performed in most of the world, except in Japan (and in a limited fashion in Korea) where early detection is often done. Thus, it continues to pose a major challenge for healthcare professionals. Risk factors for gastric cancer are Helicobacter pylori (H. pylori) infection, smoking, high salt intake, and other dietary factors.

The 5-year survival rate for curative surgical resection ranges from 30-50% for patients with stage II disease and from 10-25% for patients with stage III disease. These patients have a high likelihood of local and systemic relapse. Metastasis occurs in 80-90% of individuals with stomach cancer, with a six month survival rate of 65% in those diagnosed in early stages and less than 15% of those diagnosed in late stages.

A few gastric cancers (1% to 3%) are associated with inherited gastric cancer predisposition syndromes. E-cadherin mutations occur in approximately 25% of families with an autosomal dominant predisposition to diffuse type gastric cancers. This subset of gastric cancer has been termed hereditary diffuse gastric cancer. In these cases, genetic counseling may be provided, and to prophylactic gastrectomy in young, asymptomatic carriers of germ-line truncating may be considered.

(Harsay and Schekman, 2007) describe a novel conserved protein, Avl9p, as involved in the late secretory pathway. Phylogenetic analysis indicated evolutionary relationships between Avl9p and regulators of membrane traffic and actin function. Avl9p orthologues are found in diverse species including humans, but none of these orthologues have been previously studied.

In view of the above, there remains a strong need for new methods for the diagnosis and treatment for cancer, in particular gastric cancer. Other objects of the present invention will become apparent for the person of skill when studying the following description and the examples of the present invention.

### SUMMARY OF THE INVENTION

According to a first aspect thereof, a polypeptide is provided comprising the amino acid sequence of the protein AVL9, preferably according to SEQ ID NO: 7 according to the attached sequence listing, or a variant thereof which is at least 85% homologous to SEQ ID NO: 7, for use in medicine. In one preferred embodiment, the polypeptide consists of the amino acid sequence according to SEQ ID NO: 7 according to the attached sequence listing.

The present invention further relates to the marker protein AVL9 or a variant thereof which is at least 85% homologous to the marker protein AVL9 which can be used in the prognosis of cancer, and preferably gastric cancer. Furthermore, the present invention relates to the use of AVL9 or a variant thereof which is at least 85% homologous to AVL9 for cancer treatment. Methods of treating cancer and gastric cancer using the same are also provided. Additionally, kits comprising the same, antibodies specific for the same, and methods of using the same to generate antibodies, activated cytotoxic T lymphocytes, and/or T helper cells are also provided.

The present invention further relates to a peptide comprising at least one sequence selected from the group consisting of SEQ ID NO: 1 to SEQ ID NO: 5, or a variant thereof which is at least 85% homologous to SEQ ID NO: 1 to SEQ ID NO: 5 and induces mammalian T cells cross-reacting with said variant, wherein said peptide is not the full-length peptide of SEQ ID NO: 7, as well as nucleic acids and hosts cells encoding the same. Methods of treating cancer and gastric cancer using the same are also provided. Additionally, kits comprising the same, antibodies specific for the same, and methods of using the same to generate antibodies, activated cytotoxic T lymphocytes, and/or T helper cells are also provided.

### BRIEF DESCRIPTION OF THE FIGURES

FIG. 1 shows a quantitative peptide presentation plot illustrating the average presentation for a peptide in distinct samples visualized in a bar chart. The presentation is expressed in percent as abundance relative to the maximum area. The variation is visualized as 95% confidence intervals based on the measured replicates. If the peptide was identified in a sample but no quantification was possible, it is indicated by the label NA (not available/no area). The reason can be either a problem in the Feature finding of the LCMS run or during the normalization of the sample. Sample without detection of this peptide are marked as ND. All normal tissue samples and all samples of gastric cancer investigated are shown provided that they meet appropriate quality control criteria.

FIG. 2 shows the amino acid sequence of the protein AVL9 (SEQ ID NO: 7).

FIG. 3 shows the mRNA sequence of AVL9 (SEQ ID NO: 6).

FIG. 4 shows exemplary results of peptide-specific in vitro CD8+ T-cell responses of a healthy HLA-A*24+ donor determined by flow cytometric analysis for one peptide of the invention. CD8+ T cells were primed using artificial antigen presenting cells loaded with AVL9-001 (left panel) or irrelevant peptide IMA-xxx (right panel), respectively. After three cycles of stimulation, the detection of peptide-reactive cells was performed by double staining with AVL9-001-plus IMA-xxx A*2402-multimers. Shown cells were gated on CD8+ lymphocytes.

### DETAILED DESCRIPTION OF THE INVENTION

According to a first aspect thereof, the above object is solved by providing a polypeptide comprising the amino acid sequence of the protein AVL9, preferably according to SEQ ID NO: 7 according to the attached sequence listing, or a variant thereof which is at least 85% homologous to SEQ ID NO: 7, for use in medicine. In one preferred embodiment, the polypeptide consists of the amino acid sequence according to SEQ ID NO: 7 according to the attached sequence listing.

The present invention further relates to the marker protein AVL9 or a variant thereof which is at least 85% homologous to the marker protein AVL9 which can be used in the prognosis of cancer, and preferably gastric cancer. Furthermore, the present invention relates to the use of AVL9 or a variant thereof which is at least 85% homologous to AVL9 for cancer treatment. Methods of treating cancer and gastric cancer are also provided.

Surprisingly, the marker protein AVL9 was identified as a source protein of tumor associated antigens (TAA) according to the present invention, since only poor data is available regarding the AVL9 protein, and the biological function of the corresponding gene.

AVL9 (AP-1 Vps1 Lethal 9) was identified in a yeast genetic screen for mutations that block the late secretory pathway of eukaryotic cells. Mechanisms of exocytosis are conserved between eukaryotic cells, so results from yeast point towards corresponding mechanisms in mammals, and the majority of the components of the secretory machinery were originally identified in yeast. Earlier screens mainly identified proteins involved in ER-to-Golgi transport, because the anterograde transport from the Golgi (post-Golgi transport to other parts of the cell, including organelles and the plasma membrane) can be achieved via at least two alternative pathways. (Harsay and Schekman, 2007) used a mutant yeast strain for screening which had a block in one of the two known exocytic transport routes, so that the remaining route became essential (vps1delta-apl2delta background, lacking a dynamin and an adaptor-protein complex 1 subunit).

Upon additional depletion of Avl9p, the apl2delta-vps1delta mutant accumulated abundant structures that resembled aberrant Golgi membranes seen in mutants with blocks in exit from the Golgi. An avl9delta strain looked essentially wild type, implying a non-essential function of Avl9p. Which steps of excocytic transport are regulated by Avl9 is unknown. It might be involved in vesicle formation or recruitment of cargo. Also a role in trafficking from early endosome to late Golgi is conceivable, as it is the case for other genes like trs120, whose mutations also lead to similar phenotypes (Harsay and Schekman, 2007).

The late secretory pathway is also known to play a role in actin dynamics (Aronov and Gerst, 2004). In line with this, Avl9p was shown in a large-scale yeast interaction screen to bind the Ras-type small GTPase Rho3 that regulates the actin cytoskeleton and is partially redundant with Rho4p (Ito et al., 2001; Harsay and Schekman, 2007) were not able to confirm this interaction, but found that rho3 as well as avl9 mutations were lethal in a vps1 delta-apl2delta background, and Avl9 and all related proteins show homologies to several motifs that are also found in GTPase regulators, supporting the possibility that Avl9p may be involved in Rho3p-mediated processes, like actin organization and actin-dependent transport in the late secretory pathway. Moreover, actin distribution was perturbed in avl9 mutants. The highly polarized actin structure observed in wild type cells was lacking in the triple mutant. Over-expression of Avl9p in yeast cells did not lead to obvious effects on actin distribution, but to a defect of the late secretory pathway and growth retardation.

A large-scale interaction screen of Drosophila proteins indicates that fly Avl9 interacts with TRAF3. Therefore, Avl9 orthologos may be involved in signaling pathways which involve TRAF3, including cell survival, proliferation, and differentiation (Harsay and Schekman, 2007; Giot et al., 2003).

In a screen for small-molecule inhibitors of exocytosis in the apl2delta-vps1-delta yeast strain revealed a group of molecules able to inhibit this pathway that probably also involves Avl9. They searched for molecules which, if over-expressed, could rescue this block. Over-expression of Avl9 itself could not be tested, as earlier studies had shown the toxicity of enhanced Avl9 expression. However, they were able to show that over-expression of Ras-like GTP binding protein Gtr2 was active to rescue the exocytosis pathway. Gtr2 is known to play a role in nutrient-responsive regulator of the TORC1 signaling pathway, in exocytic cargo sorting, and epigenetic control of gene expression (Zhang et al., 2010).

The avl9 mutant was among the top-ranked hits in a genome-wide screen for mutants that are hypersensitive to both high hydrostatic pressure and cold temperature. The reason for growth retardation of avl9 mutants under these conditions is unclear. However, the data suggest that Avl9 deficiency leads to defects in traffic due to reduced membrane fluidity under these conditions. It might be that the TORC1-regulated exocytic route might be especially sensitive to conditions that reduce membrane fluidity (Abe and Minegishi, 2008; Zhang et al., 2010).

The function of Avl9 is still subject of speculation, but in addition to its cancer-relevant functions, Avl9 might be an interesting target if specifically expressed on cancer cells. As it functions in the late secretory pathway, it might be that it appears intracellularly as well as cell-surface bound.

The present invention further relates to a peptide comprising at least one sequence selected from the group consisting of SEQ ID NO: 1 to SEQ ID NO: 5, or a variant thereof which is at least 85% homologous to SEQ ID NO: 1 to SEQ ID NO: 5 and induces mammalian T cells cross-reacting with said variant, wherein said peptide is not the full-length peptide of SEQ ID NO: 7.

In the present invention, the term “homologous” refers to the degree of identity between sequences of two amino acid sequences, i.e. peptide or polypeptide sequences. The aforementioned “homology” is determined by comparing two sequences aligned under optimal conditions over the sequences to be compared. The sequences to be compared herein may have an addition or deletion (for example, gap and the like) in the optimum alignment of the two sequences. Such a sequence homology can be calculated by creating an alignment using, for example, the ClustalW algorithm (Nucleic Acid Res., 22(22): 4673 4680 (1994) or other commonly available sequence analysis software, more specifically, Vector NTI, GENETYX or analysis tools provided by public databases.

By a “variant” of the given amino acid sequence the inventors mean that the side chains of, for example, one or two of the amino acid residues are altered (for example by replacing them with the side chain of another naturally occurring amino acid residue or some other side chain) such that the peptide is still able to bind to an HLA molecule in substantially the same way as a peptide consisting of the given amino acid sequence in SEQ ID NO:1-5. For example, a peptide may be modified so that it at least maintains, if not improves, the ability to interact with and bind to the binding groove of a suitable MHC molecule, such as HLA-A*02 or -DR, and in that way it at least maintains, if not improves, the ability to bind to the TCR of activated CTL. These CTL can subsequently cross-react with cells and kill cells that express a polypeptide which contains the natural amino acid sequence of the cognate peptide as defined in the aspects of the invention. As can be derived from the scientific literature (Rammensee et al., 1997) and databases (Rammensee et al., 1999), certain positions of HLA binding peptides are typically anchor residues forming a core sequence fitting to the binding motif of the HLA receptor, which is defined by polar, electrophysical, hydrophobic and spatial properties of the polypeptide chains constituting the binding groove. Thus one skilled in the art would be able to modify the amino acid sequences set forth in SEQ ID NO:1 to SEQ ID NO:5, by maintaining the known anchor residues, and would be able to determine whether such variants maintain the ability to bind MHC class I or II molecules. The variants of the present invention retain the ability to bind to the TCR of activated CTL, which can subsequently cross-react with- and kill cells that express a polypeptide containing the natural amino acid sequence of the cognate peptide as defined in the aspects of the invention.

Those amino acid residues that do not substantially contribute to interactions with the T-cell receptor can be modified by replacement with another amino acid whose incorporation does not substantially affect T-cell reactivity and does not eliminate binding to the relevant MHC. Thus, apart from the proviso given, the peptide of the invention may be any peptide (by which term the inventors include oligopeptide or polypeptide), which includes the amino acid sequences or a portion or variant thereof as given.

It is furthermore known for MHC-class II-presented peptides that these peptides are composed of a “core sequence” having an amino acid sequence fitting to a certain HLA-allele-specific motif and, optionally, N- and/or C-terminal extensions that do not interfere with the function of the core sequence (i.e. are deemed as irrelevant for the interaction of the peptide and all or a subset of T cell clones recognizing the natural counterpart). The N- and/or C-terminal extensions can, for example, be from 1 to 10 amino acids in length, respectively. These peptides can be used either directly in order to load MHC class II molecules or the sequence can be cloned into the vectors according to the description herein below. As these peptides constitute the final product of the processing of larger peptides within the cell, longer peptides can be used as well. The peptides of the invention may be of any size, but typically they may be less than 100,000 in molecular weight, preferably less than 50,000, more preferably less than 10,000 and typically about 5,000. In terms of the number of amino acid residues, the peptides of the invention may have fewer than 1,000 residues, preferably fewer than 500 residues, more preferably fewer than 100, more preferably not more than 100 and most preferably not more than 30 residues. Accordingly, the present invention also provides peptides and variants thereof wherein said peptide or variant has an overall length of from 8 to 100, preferably from 8 to 30, and most preferred from 8 to 16, namely 8, 9, 10, 11, 12, 13, 14, 15, 16 amino acids.

For MHC class II restricted peptides, several different peptides with the same core sequence may be presented in the MHC molecule. As the interaction with the recognizing T (helper) cell is defined by a core sequence of 9 to 11 amino acids, several length variants may be recognized by the same T (helper) cell clone. Thus, several different lengths variants of a core binding sequence may be used for direct loading of MHC class II molecules without the nee for further processing and trimming at the N- or C-terminal ends. Correspondingly, naturally occurring or artificial variants that induce T cells cross-reacting with a peptide of the invention are often length variants.

If a peptide that is longer than around 12 amino acid residues is used directly to bind to a MHC class II molecule, it is preferred that the residues that flank the core HLA binding region are residues that do not substantially affect the ability of the peptide to bind specifically to the binding groove of the MHC class II molecule or to present the peptide to the T (-helper) cell. However, as already indicated above, it will be appreciated that larger peptides may be used, e.g. when encoded by a polynucleotide, since these larger peptides may be fragmented by suitable antigen-presenting cells. However, in same cases it has been shown that the core sequence flanking regions can influence the peptide binding to MHC class II molecule or the interaction of the dimeric MHC:peptide complex with the TCR in both directions compared to a reference peptide with the same core sequence. Intramolecular tertiary structures within the peptide (e.g. loop formation) normally decrease the affinities to the MHC or TCR. Intermolecular interactions of the flanking regions with parts of the MHC or TCR beside the peptide binding grooves may stabilize the interaction. These changes in affinity can have a dramatic influence on the potential of a MHC class II peptide to induce T (helper) cell responses.

It is also possible, that MHC class I epitopes, although usually from 8-10 amino acids long, are generated by peptide processing from longer peptides or proteins that include the actual epitope. It is preferred that the residues that flank the actual epitope are residues that do not substantially affect proteolytic cleavage necessary to expose the actual epitope during processing.

Accordingly, the present invention also provides peptides and variants of MHC class I epitopes wherein the peptide or variant has an overall length of from 8 to 100, preferably from 8 to 30, and most preferred from 8 to 16, namely 8, 9, 10, 11, 12, 13, 14, 15, or 16 amino acids.

The original peptides disclosed herein can be modified by the substitution of one or more residues at different, possibly selective, sites within the peptide chain, if not otherwise stated. Such substitutions may be of a conservative nature, for example, where one amino acid is replaced by an amino acid of similar structure and characteristics, such as where a hydrophobic amino acid is replaced by another hydrophobic amino acid. Even more conservative would be replacement of amino acids of the same or similar size and chemical nature, such as where leucine is replaced by isoleucine. In studies of sequence variations in families of naturally occurring homologous proteins, certain amino acid substitutions are more often tolerated than others, and these are often shown in correlation with similarities in size, charge, polarity, and hydrophobicity between the original amino acid and its replacement, and such is the basis for defining “conservative substitutions”.

Conservative substitutions are herein defined as exchanges within one of the following five groups: Group 1-small aliphatic, nonpolar or slightly polar residues (Ala, Ser, Thr, Pro, Gly); Group 2-polar, negatively charged residues and their amides (Asp, Asn, Glu, Gln); Group 3-polar, positively charged residues (His, Arg, Lys); Group 4-large, aliphatic, nonpolar residues (Met, Leu, Ile, Val, Cys); and Group 5-large, aromatic residues (Phe, Tyr, Trp).

Less conservative substitutions might involve the replacement of one amino acid by another that has similar characteristics but is somewhat different in size, such as replacement of an alanine by an isoleucine residue. Highly non-conservative replacements might involve substituting an acidic amino acid for one that is polar, or even for one that is basic in character. Such “radical” substitutions cannot, however, be dismissed as potentially ineffective since chemical effects are not totally predictable and radical substitutions might well give rise to serendipitous effects not otherwise predictable from simple chemical principles.

Of course, such substitutions may involve structures other than the common L-amino acids. Thus, D-amino acids might be substituted for the L-amino acids commonly found in the antigenic peptides of the invention and yet still be encompassed by the disclosure herein. In addition, amino acids possessing non-standard R groups (i.e., R groups other than those found in the common 20 amino acids of natural proteins) may also be used for substitution purposes to produce immunogens and immunogenic polypeptides according to the present invention.

If substitutions at more than one position are found to result in a peptide with substantially equivalent or greater antigenic activity as defined below, then combinations of those substitutions will be tested to determine if the combined substitutions result in additive or synergistic effects on the antigenicity of the peptide. At most, no more than 4 positions within the peptide would simultaneously be substituted.

The present invention provides peptides that have the ability to bind sufficiently to MHC(HLA) class I and/or II molecules for triggering an immune response of human leukocytes, especially lymphocytes, especially T lymphocytes, especially CD4-positive T lymphocytes, especially CD4-positive T lymphocytes mediating T_{H1}-type immune responses.

TABLE 1
TUMAPs derived from AVL9 according to the
present invention, SEQ ID NO: 5 is
a shortened derivative from SEQ ID NO: 1

| SEQ | | | |
|---|---|---|---|
| ID NO: | Sequence | Allele | Indication |
| 1 | FYISPVNKL | A*24 | GC, RCC, BPH (benign |
| | | | prostatic hyper- |
| | | | plasia), NSCLC, CRC |
| | | | (Colorectal carcinoma) |
| 2 | HLSDAIVEV | Likely | CCA, GC |
| | | Class I | |
| 3 | LPFLALPDGAHNY | Class I | GC |
| | | and/or | |
| | | class II | |
| 4 | LYGLLQAKL | A*24 | GC |
| 5 | YISPVNKL | Likely | GC |
| | | Class I | |

Preferred is therefore a peptide according to the present invention, wherein said peptide or variant thereof has an overall length of from 8 to 100, preferably from 8 to 30, more preferred from 8 to 16 amino acids, and most preferred wherein said peptide consists of an amino acid sequence according to any of SEQ ID NO: 1 to SEQ ID NO: 5.

Further preferred is therefore a peptide or variant thereof according to the present invention, wherein said peptide or variant thereof has the ability to bind to a molecule of the human major histocompatibility complex (MHC) class-I and/or -II.

In the present invention, the inventors isolated and characterized peptides binding to HLA class I or II molecules directly from mammalian tumors, i.e. primary samples of mainly gastric cancer patients, but also from primary tissue samples of gastric cancer, colorectal cancers, renal cell carcinoma, lung cancers, pancreatic cancers, malignant melanoma, and cancer of the stomach.

As described herein below, the peptides that form the basis of the present invention have all been identified as presented by MHC class I or II bearing cells. Thus, these particular peptides as well as other peptides containing the sequence (i.e. derived peptides) all elicit a specific T-cell response, although the extent to which such response will be induced might vary from individual peptide to peptide and from individual patient to patient. Differences, for example, could be caused due to mutations in the peptides. The person of skill in the present art is well aware of methods that can be applied to determine the extent to which a response is induced by an individual peptide, in particular with reference to the examples herein and the respective literature.

Further preferred is therefore a peptide according to the present invention, wherein said peptide is capable of stimulating CD4 or CD8 T cells. Preferably the variants of the invention will induce T-cells cross-reacting with the respective peptide of the invention.

In a particularly preferred embodiment of the invention the peptide consists or consists essentially of an amino acid sequence according to SEQ ID NO: 1 to SEQ ID NO: 5. “Consisting essentially of” shall mean that a peptide according to the present invention, in addition to the sequence according to any of SEQ ID NO: 1 to SEQ ID NO: 5 or a variant thereof contains additional N- and/or C-terminally located stretches of amino acids that are not necessarily forming part of the peptide that functions as an epitope for MHC molecules epitope. Nevertheless, these stretches can be important to provide an efficient introduction of the peptide according to the present invention into the cells. In one embodiment of the present invention, the peptide is a fusion protein which comprises, for example, the 80 N-terminal amino acids of the HLA-DR antigen-associated invariant chain (p33, in the following “Ii”) as derived from the NCBI, GenBank Accession-number X00497 (Strubin et al., 1984).

In addition, the present invention further provides a peptide according to the present invention as described herein, wherein said peptide comprises chemically modified amino acids, and/or includes non-peptide bonds.

In addition, the peptide or variant may be modified further to improve stability and/or binding to MHC molecules in order to elicit a stronger immune response. Methods for such an optimization of a peptide sequence are well known in the art and include, for example, the introduction of reverse peptide bonds or non-peptide bonds.

In a reverse peptide bond amino acid residues are not joined by peptide (—CO—NH—) linkages but the peptide bond is reversed. Such retro-inverso peptidomimetics may be made using methods known in the art, for example such as those described in Meziere et al (1997) J. Immunol. 159, 3230-3237, incorporated herein by reference. This approach involves making pseudopeptides containing changes involving the backbone, and not the orientation of side chains. (Meziere et al., 1997) show that for MHC binding and T helper cell responses, these pseudopeptides are useful. Retro-inverse peptides, which contain NH—CO bonds instead of CO—NH peptide bonds, are much more resistant to proteolysis.

A non-peptide bond is, for example, —CH₂—NH, —CH₂S—, —CH₂CH₂—, —CH═CH—, —COCH₂—, —CH(OH)CH₂—, and —CH₂SO—. U.S. Pat. No. 4,897,445 provides a method for the solid phase synthesis of non-peptide bonds (—CH₂—NH) in polypeptide chains which involves polypeptides synthesized by standard procedures and the non-peptide bond synthesized by reacting an amino aldehyde and an amino acid in the presence of NaCNBH₃.

Peptides comprising the sequences described above may be synthesized with additional chemical groups present at their amino and/or carboxy termini, to enhance the stability, bioavailability, and/or affinity of the peptides. For example, hydrophobic groups such as carbobenzoxyl, dansyl, or t-butyloxycarbonyl groups may be added to the peptides' amino termini. Likewise, an acetyl group or a 9-fluorenylmethoxy-carbonyl group may be placed at the peptides' amino termini. Additionally, the hydrophobic group, t-butyloxycarbonyl, or an amido group may be added to the peptides' carboxy termini.

Further, the peptides of the invention may be synthesized to alter their steric configuration. For example, the D-isomer of one or more of the amino acid residues of the peptide may be used, rather than the usual L-isomer. Still further, at least one of the amino acid residues of the peptides of the invention may be substituted by one of the well known non-naturally occurring amino acid residues. Alterations such as these may serve to increase the stability, bioavailability and/or binding action of the peptides of the invention.

Similarly, a peptide or variant of the invention may be modified chemically by reacting specific amino acids either before or after synthesis of the peptide. Examples for such modifications are well known in the art and are summarized e.g. in R. Lundblad, Chemical Reagents for Protein Modification, 3rd ed. CRC Press, 2005, which is incorporated herein by reference. Chemical modification of amino acids includes but is not limited to, modification by acylation, amidination, pyridoxylation of lysine, reductive alkylation, trinitrobenzylation of amino groups with 2,4,6-trinitrobenzene sulphonic acid (TNBS), amide modification of carboxyl groups and sulphydryl modification by performic acid oxidation of cysteine to cysteic acid, formation of mercurial derivatives, formation of mixed disulphides with other thiol compounds, reaction with maleimide, carboxymethylation with iodoacetic acid or iodoacetamide and carbamoylation with cyanate at alkaline pH, although without limitation thereto. In this regard, the skilled person is referred to Chapter 15 of Current Protocols In Protein Science, Eds. Coligan et al. (John Wiley & Sons NY 1995-2000) for more extensive methodology relating to chemical modification of proteins.

Briefly, modification of e.g. arginyl residues in proteins is often based on the reaction of vicinal dicarbonyl compounds such as phenylglyoxal, 2,3-butanedione, and 1,2-cyclohexanedione to form an adduct. Another example is the reaction of methylglyoxal with arginine residues. Cysteine can be modified without concomitant modification of other nucleophilic sites such as lysine and histidine. As a result, a large number of reagents are available for the modification of cysteine. The websites of companies such as Sigma-Aldrich (http://www.sigma-aldrich.com) provide information on specific reagents.

Selective reduction of disulfide bonds in proteins is also common. Disulfide bonds can be formed and oxidized during the heat treatment of biopharmaceuticals.

Woodward's Reagent K may be used to modify specific glutamic acid residues. N-(3-(dimethylamino)propyl)-N′-ethylcarbodiimide can be used to form intra-molecular crosslinks between a lysine residue and a glutamic acid residue.

For example, diethylpyrocarbonate is a reagent for the modification of histidyl residues in proteins. Histidine can also be modified using 4-hydroxy-2-nonenal.

The reaction of lysine residues and other α-amino groups is, for example, useful in binding of peptides to surfaces or the cross-linking of proteins/peptides. Lysine is the site of attachment of poly(ethylene)glycol and the major site of modification in the glycation of proteins.

Methionine residues in proteins can be modified with e.g. iodoacetamide, bromoethylamine, and chloramine T.

Tetranitromethane and N-acetylimidazole can be used for the modification of tyrosyl residues. Cross-linking via the formation of dityrosine can be accomplished with hydrogen peroxide/copper ions.

Recent studies on the modification of tryptophan have used N-bromosuccinimide, 2-hydroxy-5-nitrobenzyl bromide or 3-bromo-3-methyl-2-(2-nitrophenylmercapto)-3H-indole (BPNS-skatole).

Successful modification of therapeutic proteins and peptides with PEG is often associated with an extension of circulatory half-life while cross-linking of proteins with glutaraldehyde, polyethyleneglycol diacrylate and formaldehyde is used for the preparation of hydrogels. Chemical modification of allergens for immunotherapy is often achieved by carbamylation with potassium cyanate.

A peptide or variant, wherein the peptide is modified or includes non-peptide bonds is a preferred embodiment of the invention. Generally, peptides and variants (at least those containing peptide linkages between amino acid residues) may be synthesized by the Fmoc-polyamide mode of solid-phase peptide synthesis as disclosed by (Lu et al., 1981) and references therein. Temporary N-amino group protection is afforded by the 9-fluorenylmethyloxycarbonyl (Fmoc) group. Repetitive cleavage of this highly base-labile protecting group is done using 20% piperidine in N,N-dimethylformamide. Side-chain functionalities may be protected as their butyl ethers (in the case of serine threonine and tyrosine), butyl esters (in the case of glutamic acid and aspartic acid), butyloxycarbonyl derivative (in the case of lysine and histidine), trityl derivative (in the case of cysteine) and 4-methoxy-2,3,6-trimethylbenzenesulphonyl derivative (in the case of arginine). Where glutamine or asparagine are C-terminal residues, use is made of the 4,4′-dimethoxybenzhydryl group for protection of the side chain amido functionalities. The solid-phase support is based on a polydimethyl-acrylamide polymer constituted from the three monomers dimethylacrylamide (backbone-monomer), bisacryloylethylene diamine (cross linker) and acryloylsarcosine methyl ester (functionalizing agent). The peptide-to-resin cleavable linked agent used is the acid-labile 4-hydroxymethyl-phenoxyacetic acid derivative. All amino acid derivatives are added as their preformed symmetrical anhydride derivatives with the exception of asparagine and glutamine, which are added using a reversed N,N-dicyclohexylcarbodiimide/1hydroxybenzotriazole mediated coupling procedure. All coupling and deprotection reactions are monitored using ninhydrin, trinitrobenzene sulphonic acid or isotin test procedures. Upon completion of synthesis, peptides are cleaved from the resin support with concomitant removal of side-chain protecting groups by treatment with 95% trifluoroacetic acid containing a 50% scavenger mix. Scavengers commonly used include ethandithiol, phenol, anisole and water, the exact choice depending on the constituent amino acids of the peptide being synthesized. Also a combination of solid phase and solution phase methodologies for the synthesis of peptides is possible (see, for example, (Bruckdorfer et al., 2004)) and the references as cited therein).

Trifluoroacetic acid is removed by evaporation in vacuo, with subsequent trituration with diethyl ether affording the crude peptide. Any scavengers present are removed by a simple extraction procedure which on lyophilization of the aqueous phase affords the crude peptide free of scavengers. Reagents for peptide synthesis are generally available from e.g. Calbiochem-Novabiochem (UK) Ltd, Nottingham NG7 2QJ, UK.

Purification may be performed by any one, or a combination of, techniques such as recrystallization, size exclusion chromatography, ion-exchange chromatography, hydrophobic interaction chromatography and (usually) reverse-phase high performance liquid chromatography using e.g. acetonitrile/water gradient separation.

In addition, the present invention further provides chimeric/fusion proteins/peptides comprising the AVL9 polypeptides, and fragments thereof, including functional, proteolytic and antigenic fragments.

The fusion partner or sections of a hybrid molecule suitably provide epitopes that stimulate CD4⁺ T-cells. CD4⁺ stimulating epitopes are well known in the art and include those identified in tetanus toxoid. In a further preferred embodiment the peptide is a fusion protein, in particular comprising N-terminal amino acids of the HLA-DR antigen-associated invariant chain (Ii). In one embodiment the peptide of the invention is a truncated human protein or a fusion protein of a protein fragment and another polypeptide portion provided that the human portion includes one or more inventive amino acid sequences. Preferred is therefore a peptide according to the present invention, wherein said peptide is part of a fusion protein, in particular comprising N-terminal amino acids of the HLA-DR antigen-associated invariant chain (Ii).

Another aspect of the present invention then relates to a nucleic acid, encoding for a peptide according to the present invention, or an expression vector capable of expressing said nucleic acid.

The nucleic acid may be, for example, DNA, cDNA, PNA, CNA, RNA or combinations thereof, either single- and/or double-stranded, or native or stabilized forms of polynucleotides, such as, for example, polynucleotides with a phosphorothioate backbone and it may or may not contain introns so long as it codes for the peptide. Of course, only peptides that contain naturally occurring amino acid residues joined by naturally occurring peptide bonds are encodable by a polynucleotide. A still further aspect of the invention provides an expression vector capable of expressing a polypeptide according to the invention.

A variety of methods have been developed to link polynucleotides, especially DNA, to vectors for example via complementary cohesive termini. For instance, complementary homopolymer tracts can be added to the DNA segment to be inserted to the vector DNA. The vector and DNA segment are then joined by hydrogen bonding between the complementary homopolymeric tails to form recombinant DNA molecules.

Synthetic linkers containing one or more restriction sites provide an alternative method of joining the DNA segment to vectors. Synthetic linkers containing a variety of restriction endonuclease sites are commercially available from a number of sources including International Biotechnologies Inc, New Haven, Conn., USA.

A desirable method of modifying the DNA encoding the polypeptide of the invention employs the polymerase chain reaction as disclosed by (Saiki et al., 1988). This method may be used for introducing the DNA into a suitable vector, for example by engineering in suitable restriction sites, or it may be used to modify the DNA in other useful ways as is known in the art. If viral vectors are used, pox- or adenovirus vectors are preferred.

The DNA (or in the case of retroviral vectors, RNA) may then be expressed in a suitable host to produce a polypeptide comprising the peptide or variant of the invention. Thus, the DNA encoding the peptide or variant of the invention may be used in accordance with known techniques, appropriately modified in view of the teachings contained herein, to construct an expression vector, which is then used to transform an appropriate host cell for the expression and production of the polypeptide of the invention. Such techniques include those disclosed in U.S. Pat. Nos. 4,440,859, 4,530,901, 4,582,800, 4,677,063, 4,678,751, 4,704,362, 4,710,463, 4,757,006, 4,766,075, and 4,810,648.

The DNA (or in the case of retroviral vectors, RNA) encoding the polypeptide constituting the compound of the invention may be joined to a wide variety of other DNA sequences for introduction into an appropriate host. The companion DNA will depend upon the nature of the host, the manner of the introduction of the DNA into the host, and whether episomal maintenance or integration is desired.

Generally, the DNA is inserted into an expression vector, such as a plasmid, in proper orientation and correct reading frame for expression. If necessary, the DNA may be linked to the appropriate transcriptional and translational regulatory control nucleotide sequences recognized by the desired host, although such controls are generally available in the expression vector. The vector is then introduced into the host through standard techniques. Generally, not all of the hosts will be transformed by the vector. Therefore, it will be necessary to select for transformed host cells. One selection technique involves incorporating into the expression vector a DNA sequence, with any necessary control elements, that codes for a selectable trait in the transformed cell, such as antibiotic resistance.

Alternatively, the gene for such selectable trait can be on another vector, which is used to co-transform the desired host cell.

In yet another embodiment of the present invention, the nucleic acid encodes a human AVL9 protein. In a particular embodiment, the nucleic acid that encodes the human AVL9 comprises the nucleotide sequence of SEQ ID NO: 6. In another embodiment, the nucleic acid encodes for a human protein comprising the amino acid sequence of SEQ ID NO: 7 and including or comprising 1 to 10 conservative amino acid substitutions.

All of the nucleic acids of the present invention can further comprise a heterologous nucleotide sequence. In addition, recombinant DNA molecules that are operatively linked to an expression control sequence can be constructed from and/or derived from the nucleic acids of the present invention. In addition, cells that have been transfected and/or transformed with the expression vectors of the present invention, in which the AVL9 protein is expressed by the cell are also part of the present invention. In a preferred embodiment, the cell is a mammalian cell.

The present invention also provides methods of expressing the recombinant AVL9 polypeptides and fragments thereof in cells containing the expression vectors of the present invention. One such method comprises culturing the cell in an appropriate cell culture medium under conditions that provide for expression of the recombinant polypeptide (e.g., AVL9) by the cell. In a preferred embodiment, the method further comprises the step of purifying the recombinant AVL9. The purified form of the recombinant AVL9 is also part of the present invention.

The present invention further provides nucleic acids that hybridize under standard conditions to a nucleic acid of the present invention.

In a preferred embodiment, the nucleic acid encodes an AVL9 polypeptide that comprises a nucleus localization signal and/or a glutamine rich region. Preferably, the nucleic acid encodes a AVL9 that is localized in the nuclei.

In another embodiment, the nucleic acid encodes for an AVL9 polypeptide having an apoptosis-inducing domain (e.g., the protein and/or a fragment thereof can induce apoptosis in a cell). In another embodiment, the nucleic acid encodes an AVL9 polypeptide that has a transactivation domain.

Another aspect of the present invention then relates to a pharmaceutical composition, comprising at least one of an AVL9 polypeptide according to the present invention, at least one of a peptide according to the present invention, or at least one of a nucleic acid or expression vector according to the present invention, together with suitable pharmaceutical auxiliary agents.

For this, the polypeptides, peptides and optionally other molecules (such as, for example, antibodies or other anti-cancer agents) are dissolved or suspended in a pharmaceutically acceptable, preferably aqueous carrier. In addition, the composition can contain excipients, such as buffers, binding agents, blasting agents, diluents, flavors, lubricants, etc. The peptides can also be administered together with immune stimulating substances, such as cytokines Exemplary formulations can be found in EP2113253. An extensive listing of excipients that can be used in such a composition, can be, for example, taken from A. Kibbe, Handbook of Pharmaceutical Excipients, 3. Ed. 2000, American Pharmaceutical Association and pharmaceutical press. The composition can be used for the prevention, prophylaxis and/or therapy of proliferative and/or cancerous diseases as described herein.

Preferably, this pharmaceutical composition is used for parenteral administration, such as subcutaneous, intradermal, intramuscular or oral administration.

In a particularly preferred embodiment, the pharmaceutical composition according the present invention is an anti-cancer vaccine, optionally containing at least one additional peptide having a sequence selected from the group consisting of any of SEQ ID NO: 8 to SEQ ID NO: 47.

Preferably, the medicament of the present invention is a vaccine. It may be administered directly into the patient, into the affected organ or systemically i.d., i.m., s.c., i.p. and i.v., or applied ex vivo to cells derived from the patient or a human cell line which are subsequently administered to the patient, or used in vitro to select a subpopulation of immune cells derived from the patient, which are then re-administered to the patient. If the nucleic acid is administered to cells in vitro, it may be useful for the cells to be transfected so as to co-express immune-stimulating cytokines, such as interleukin-2 The peptide may be substantially pure, or combined with an immune-stimulating adjuvant (see below) or used in combination with immune-stimulatory cytokines, or be administered with a suitable delivery system, for example liposomes. The peptide may also be conjugated to a suitable carrier such as keyhole limpet haemocyanin (KLH) or mannan (see WO 95/18145 and (Longenecker et al., 1993)). The peptide may also be tagged, may be a fusion protein, or may be a hybrid molecule. The peptides whose sequence is given in the present invention are expected to stimulate CD4 or CD8 T cells. However, stimulation of CD8 CTLs is more efficient in the presence of help provided by CD4 T-helper cells. Thus, for MHC Class I epitopes that stimulate CD8 CTL the fusion partner or sections of a hybrid molecule suitably provide epitopes which stimulate CD4-positive T cells. CD4- and CD8-stimulating epitopes are well known in the art and include those identified in the present invention.

In one aspect, the vaccine comprises at least one peptide having the amino acid sequence set forth in SEQ ID NO:1, 2, 3, 4 or 5, and at least one additional peptide, preferably two to 50, more preferably two to 25, even more preferably two to 15 and most preferably two, three, four, five, six, seven, eight, nine, ten, eleven, twelve or thirteen peptides. The peptide(s) may be derived from one or more specific TAAs and may bind to MHC class I and/or class II molecules. Preferably the at least one additional peptide has the amino acid sequence set forth in SEQ ID NO: 8 to SEQ ID NO: 47 as shown in the following tables.

TABLE 2
Preferred additional immunogenic peptides useful
in a composition of the invention

| SEQ | | | | |
|---|---|---|---|---|
| ID | | | Gene | binds |
| NO | Peptide ID | Sequence | Symbol | to MHC |
| 8 | CDC2-001 | LYQILQGIVF | CDK1 | HLA-A*024 |
| 9 | ASPM-002 | SYNPLWLRI | ASPM | HLA-A*024 |
| 10 | UCHL5-001 | NYLPFIMEL | UCHL5 | HLA-A*024 |
| 11 | MET-006 | SYIDVLPEF | MET | HLA-A*024 |
| 12 | PROM1-001 | SYIIDPLNL | PROM1 | HLA-A*024 |
| 13 | UQCRB-001 | YYNAAGFNKL | UQCRB | HLA-A*024 |
| 14 | MST1R-001 | NYLLYVSNF | MST1R | HLA-A*024 |
| 15 | PPAP2C-001 | AYLVYTDRL | PPAP2C | HLA-A*024 |
| 16 | SMC4-001 | HYKPTPLYF | SMC4 | HLA-A*024 |
| 17 | MMP11-001 | VWSDVTPLTF | MMP11 | HLA-A*024 |
| 18 | BIR-002 | TLGEFLKLDRERAKN | BIRC5 | HLA-DR |
| | | | | and |
| | | | | HLA-A*02 |
| 19 | CDC42-001 | DDPSTIEKLAKNKQKP | CDC42 | HLA-DR |
| 20 | CDC42-002 | NKQKPITPETAEKLARD | CDC42 | HLA-DR |
| 21 | SPP1-001 | NGAYKAIPVAQDLNAPS | SPP1 | HLA-DR |
| 22 | BIR-002a | TLGEFLKLDRERAKD | Survivin | HLA-DR |
| | | | | and |
| | | | | HLA-A*02 |
| 23 | BIR-002b | FTELTLGEF | Survivin | HLA-A1 |
| 24 | BIR-002c | LMLGEFLKL | Survivin | HLA-A2 |
| 25 | BIR-002d | EPDLAQCFY | Survivin | HLA-B35 |
| 26 | NUF2-001 | VYGIRLEHF | NUF2 | HLA-A*024 |
| 27 | ABL1-001 | TYGNLLDYL | ABL1 | HLA-A*024 |
| 28 | NUF2-002 | RFLSGIINF | NUF2 | HLA-A*024 |

TABLE 3
Additional immunogenic peptides useful in a
composition of the invention

| SEQ | | | Source |
|---|---|---|---|
| ID NO: | Peptide Code | Sequence | Protein(s) |
| 29 | NFYB-001 | VYTTSYQQI | NFYB |
| 30 | MUC6-001 | NYEETFPHI | MUC6 |
| 31 | ASPM-001 | RYLWATVTI | ASPM |
| 32 | EPHA2-005 | VYFSKSEQL | EPHA2 |
| 33 | MMP3-001 | VFIFKGNQF | MMP3 |
| 34 | PLK4-001 | QYASRFVQL | PLK4 |
| 35 | ATAD2-002 | KYLTVKDYL | ATAD2 |
| 36 | COL12A1-001 | VYNPTPNSL | COL12A1 |
| 37 | COL6A3-001 | SYLQAANAL | COL6A3 |
| 38 | FANCI-001 | FYQPKIQQF | FANCI |
| 39 | RPS11-001 | YYKNIGLGF | RPS11 |
| 40 | ATAD2-001 | AYAIIKEEL | ATAD2 |
| 41 | ATAD2-003 | LYPEVFEKF | ATAD2 |
| 42 | HSP90B1-001 | KYNDTFWKEF | HSP90B1 |
| 43 | SIAH2-001 | VFDTAIAHLF | SIAH2 |
| 44 | SLC6A6-001 | VYPNWAIGL | SLC6A6 |
| 45 | IQGAP3-001 | VYKVVGNLL | IQGAP3 |
| 46 | ERBB3-001 | VYIEKNDKL | ERBB3 |
| 47 | KIF2C-001 | IYNGKLFDLL | KIF2C |

The vaccine of the invention may also include one or more adjuvants. Adjuvants are substances that non-specifically enhance or potentiate the immune response (e.g., immune responses mediated by CTLs and helper-T (T_{H}) cells to an antigen, and would thus be considered useful in the medicament of the present invention. Suitable adjuvants include, but are not limited to, 1018 ISS, aluminum salts, Amplivax®, AS15, BCG, CP-870,893, CpG7909, CyaA, dSLIM, flagellin or TLR5 ligands derived from flagellin, FLT3 ligand, GM-CSF, IC30, IC31, Imiquimod (ALDARA®), resiquimod, ImuFact IMP321, Interleukins as IL-2, IL-13, IL-21, Interferon-alpha or -beta, or pegylated derivatives thereof, IS Patch, ISS, ISCOMATRIX, ISCOMs, JuvImmune, LipoVac, MALP2, MF59, monophosphoryl lipid A, Montanide IMS 1312, Montanide ISA 206, Montanide ISA 50V, Montanide ISA-51, water-in-oil and oil-in-water emulsions, OK-432, OM-174, OM-197-MP-EC, ONTAK, OspA, PepTel® vector system, poly(lactid co-glycolid) [PLG]-based and dextran microparticles, talactoferrin SRL172, Virosomes and other Virus-like particles, YF-17D, VEGF trap, R848, beta-glucan, Pam3Cys, Aquila's QS21 stimulon, which is derived from saponin, mycobacterial extracts and synthetic bacterial cell wall mimics, and other proprietary adjuvants such as Ribi's Detox, Quil, or Superfos. Adjuvants such as Freund's or GM-CSF are preferred. Several immunological adjuvants (e.g., MF59) specific for dendritic cells and their preparation have been described previously (Allison and Krummel, 1995; Allison and Krummel, 1995). Also cytokines may be used. Several cytokines have been directly linked to influencing dendritic cell migration to lymphoid tissues (e.g., TNF-), accelerating the maturation of dendritic cells into efficient antigen-presenting cells for T-lymphocytes (e.g., GM-CSF, IL-1 and IL-4) (U.S. Pat. No. 5,849,589, specifically incorporated herein by reference in its entirety) and acting as immunoadjuvants (e.g., IL-12, IL-15, IL-23, IL-7, IFN-alpha. IFN-beta) (Gabrilovich et al., 1996).

CpG immunostimulatory oligonucleotides have also been reported to enhance the effects of adjuvants in a vaccine setting. Without being bound by theory, CpG oligonucleotides act by activating the innate (non-adaptive) immune system via Toll-like receptors (TLR), mainly TLR9. CpG triggered TLR9 activation enhances antigen-specific humoral and cellular responses to a wide variety of antigens, including peptide or protein antigens, live or killed viruses, dendritic cell vaccines, autologous cellular vaccines and polysaccharide conjugates in both prophylactic and therapeutic vaccines. More importantly it enhances dendritic cell maturation and differentiation, resulting in enhanced activation of T_{H1} cells and strong cytotoxic T-lymphocyte (CTL) generation, even in the absence of CD4 T cell help. The T_{H1} bias induced by TLR9 stimulation is maintained even in the presence of vaccine adjuvants such as alum or incomplete Freund's adjuvant (IFA) that normally promote a T_{H2} bias. CpG oligonucleotides show even greater adjuvant activity when formulated or co-administered with other adjuvants or in formulations such as microparticles, nanoparticles, lipid emulsions or similar formulations, which are especially necessary for inducing a strong response when the antigen is relatively weak. They also accelerate the immune response and enable the antigen doses to be reduced by approximately two orders of magnitude, with comparable antibody responses to the full-dose vaccine without CpG in some experiments (Krieg, 2006). U.S. Pat. No. 6,406,705 B1 describes the combined use of CpG oligonucleotides, non-nucleic acid adjuvants and an antigen to induce an antigen-specific immune response. A CpG TLR9 antagonist is dSLIM (double Stem Loop Immunomodulator) by Mologen (Berlin, Germany) which is a preferred component of the pharmaceutical composition of the present invention. Other TLR binding molecules such as RNA binding TLR 7, TLR 8 and/or TLR 9 may also be used.

Other examples for useful adjuvants include, but are not limited to chemically modified CpGs (e.g. CpR, Idera), dsRNA analogues such as Poly(I:C) and derivates thereof (e.g. AmpliGen®, Hiltonol®, poly-(ICLC), poly(IC-R), poly(I:C12U), non-CpG bacterial DNA or RNA as well as immunoactive small molecules and antibodies such as cyclophosphamide, sunitinib, Bevacizumab, celebrex, NCX-4016, sildenafil, tadalafil, vardenafil, sorafenib, temozolomide, temsirolimus, XL-999, CP-547632, pazopanib, VEGF Trap, ZD2171, AZD2171, anti-CTLA4, other antibodies targeting key structures of the immune system (e.g. anti-CD40, anti-TGFbeta, anti-TNFalpha receptor) and SC58175, which may act therapeutically and/or as an adjuvant. The amounts and concentrations of adjuvants and additives useful in the context of the present invention can readily be determined by the skilled artisan without undue experimentation.

Preferred adjuvants are imiquimod, resiquimod, GM-CSF, cyclophosphamide, sunitinib, bevacizumab, interferon-alpha, CpG oligonucleotides and derivates, poly-(I:C) and derivates, RNA, sildenafil, and particulate formulations with PLG or virosomes.

In a preferred embodiment, the pharmaceutical composition according to the invention the adjuvant is selected from the group consisting of colony-stimulating factors, such as Granulocyte Macrophage Colony Stimulating Factor (GM-CSF, sargramostim), imiquimod, resiquimod, and interferon-alpha.

In a preferred embodiment, the pharmaceutical composition according to the invention the adjuvant is selected from the group consisting of colony-stimulating factors, such as Granulocyte Macrophage Colony Stimulating Factor (GM-CSF, sargramostim), imiquimod and resimiquimod.

In a preferred embodiment of the pharmaceutical composition according to the invention, the adjuvant is imiquimod or resiquimod.

Another aspect of the present invention then relates to an antibody which specifically binds to a peptide according to the present invention as described herein. The antibodies of the present invention can be polyclonal antibodies, monoclonal antibodies and/or chimeric antibodies. Immortal cell lines that produce a monoclonal antibody of the present invention are also part of the present invention. Antibodies, preferably binding specifically to the AVL9 polypeptides, to the chimeric/fusion proteins comprising the AVL9 polypeptides (e.g. according to SEQ ID NO: 7) as described herein, as well as to the fragments of the AVL9 polypeptides, such as, for example, the peptides as set forth in SEQ ID NO: 1, 2, 3, 4 or 5, including proteolytic, and antigenic fragments, and to the chimeric/fusion proteins/peptides comprising these fragments are also part of the present invention. In addition, methods of using such antibodies for the prognosis of proliferative diseases, cancer, and gastric cancer in particular, are also part of the present invention.

In another aspect thereof, the present invention relates to an in vitro method for producing activated cytotoxic T lymphocytes (CTL), comprising contacting in vitro CTL with antigen loaded human class I MHC molecules expressed on the surface of a suitable antigen-presenting cell or an artificial construct mimicking an antigen-presenting cell for a period of time sufficient to activate said CTL in an antigen specific manner, wherein said antigen is a peptide according to any of SEQ ID NO: 1 to SEQ ID NO: 5 of the present invention.

A number of methods may be used for generating CTL in vitro. For example, the methods described in (Peoples et al., 1995) and (Kawakami et al., 1992) use autologous tumor-infiltrating lymphocytes in the generation of CTL. (Plebanski et al., 1995) makes use of autologous peripheral blood lymphocytes (PLBs) in the preparation of CTL. (Jochmus et al., 1997) describes the production of autologous CTL by pulsing dendritic cells with peptide or polypeptide, or via infection with recombinant virus. (Hill et al., 1995) and (Jerome et al., 1993) make use of B cells in the production of autologous CTL. In addition, macrophages pulsed with peptide or polypeptide, or infected with recombinant virus, may be used in the preparation of autologous CTL. (Walter et al., 2003) describe the in vitro priming of T cells by using artificial antigen presenting cells (aAPCs), which is also a suitable way for generating T cells against the peptide of choice. In this study, aAPCs were generated by the coupling of preformed MHC:peptide complexes to the surface of polystyrene particles (microbeads) by biotin:streptavidin biochemistry. This system permits the exact control of the MHC density on aAPCs, which allows to selectively elicit high- or low-avidity antigen-specific T cell responses with high efficiency from blood samples. Apart from MHC:peptide complexes, aAPCs should carry other proteins with co-stimulatory activity like anti-CD28 antibodies coupled to their surface. Furthermore such aAPC-based systems often require the addition of appropriate soluble factors, e.g. cytokines like interleukin-12.

Allogeneic cells may also be used in the preparation of T cells and a method is described in detail in WO 97/26328, incorporated herein by reference. For example, in addition to Drosophila cells and T2 cells, other cells may be used to present antigens such as CHO cells, baculovirus-infected insect cells, bacteria, yeast, vaccinia-infected target cells. In addition plant viruses may be used (see, for example, (Porta et al., 1994)) which describes the development of cowpea mosaic virus as a high-yielding system for the presentation of foreign peptides.

The activated T cells that are directed against the peptides of the invention are useful in therapy. Thus, a further aspect of the invention provides activated T cells obtainable by the foregoing methods of the invention.

Activated T cells, which are produced by the above method, will selectively recognize a cell that aberrantly expresses a polypeptide that comprises an amino acid sequence of SEQ ID NO: 1 to 5.

Preferably, the T cell recognizes the cell by interacting through its TCR with the HLA/peptide-complex (for example, binding). The T cells are useful in a method of killing target cells in a patient whose target cells aberrantly express a polypeptide comprising an amino acid sequence of the invention wherein the patient is administered an effective number of the activated T cells. The T cells that are administered to the patient may be derived from the patient and activated as described above (i.e. they are autologous T cells). Alternatively, the T cells are not from the patient but are from another individual. Of course, it is preferred if the individual is a healthy individual. By “healthy individual” the inventors mean that the individual is generally in good health, preferably has a competent immune system and, more preferably, is not suffering from any disease which can be readily tested for, and detected.

In vivo, the target cells for the CD4-positive T cells according to the present invention can be cells of the tumor (which sometimes express MHC class II) and/or stromal cells surrounding the tumor (tumor cells) (which sometimes also express MHC class II; (Dengjel et al., 2006)).

The T cells of the present invention may be used as active ingredients of a therapeutic composition. Thus, the invention also provides a method of killing target cells in a patient whose target cells aberrantly express a polypeptide comprising an amino acid sequence of the invention, the method comprising administering to the patient an effective number of T cells as defined above.

By “aberrantly expressed” the inventors mean that the polypeptide is over-expressed compared to normal levels of expression or that the gene is silent in the tissue from which the tumor is derived but in the tumor it is expressed. By “over-expressed” the inventors mean that the polypeptide is present at a level at least 1.2-fold of that present in normal tissue; preferably at least 2-fold, and more preferably at least 5-fold or 10-fold the level present in normal tissue. T cells may be obtained by methods known in the art, e.g. those described herein.

Protocols for this so-called adoptive transfer of T cells are well known in the art and can be found, e.g. in (Rosenberg et al., 1987; Rosenberg et al., 1988; Dudley et al., 2002; Yee et al., 2002; Dudley et al., 2005); reviewed in (Gattinoni et al., 2006) and (Morgan et al., 2006).

Another aspect of the present invention then relates to an activated cytotoxic T lymphocyte (CTL) which selectively recognizes a cell which aberrantly expresses a polypeptide comprising an amino acid sequence according to the present invention (i.e. usually AVL9-derived).

Yet another aspect of the present invention then relates to a host cell comprising a recombinant nucleic acid or the expression vector according to the present invention, such as, for example, an antigen presenting cell, a dendritic cell or an antigen presenting cell.

Host cells that have been transformed by the recombinant DNA of the invention are then cultured for a sufficient time and under appropriate conditions known to those skilled in the art in view of the teachings disclosed herein to permit the expression of the polypeptide, which can then be recovered.

Many expression systems are known, including bacteria (for example E. coli and Bacillus subtilis), yeasts (for example Saccharomyces cerevisiae), filamentous fungi (for example Aspergillus spec.), plant cells, animal cells and insect cells. Preferably, the system can be mammalian cells such as CHO cells available from the ATCC Cell Biology Collection.

A typical mammalian cell vector plasmid for constitutive expression comprises the CMV or SV40 promoter with a suitable poly A tail and a resistance marker, such as neomycin. One example is pSVL available from Pharmacia, Piscataway, N.J., USA. An example of an inducible mammalian expression vector is pMSG, also available from Pharmacia. Useful yeast plasmid vectors are pRS403-406 and pRS413-416 and are generally available from Stratagene Cloning Systems, La Jolla, Calif. 92037, USA. Plasmids pRS403, pRS404, pRS405 and pRS406 are Yeast Integrating plasmids (YIps) and incorporate the yeast selectable markers HIS3, TRP1, LEU2 and URA3. Plasmids pRS413-416 are Yeast Centromere plasmids (Ycps). CMV promoter-based vectors (for example from Sigma-Aldrich) provide transient or stable expression, cytoplasmic expression or secretion, and N-terminal or C-terminal tagging in various combinations of FLAG, 3×FLAG, c-myc or MAT. These fusion proteins allow for detection, purification and analysis of recombinant protein. Dual-tagged fusions provide flexibility in detection.

The strong human cytomegalovirus (CMV) promoter regulatory region drives constitutive protein expression levels as high as 1 mg/L in COS cells. For less potent cell lines, protein levels are typically ˜0.1 mg/L. The presence of the SV40 replication origin will result in high levels of DNA replication in SV40 replication permissive COS cells. CMV vectors, for example, can contain the pMB1 (derivative of pBR322) origin for replication in bacterial cells, the b-lactamase gene for ampicillin resistance selection in bacteria, hGH polyA, and the f1 origin. Vectors containing the preprotrypsin leader (PPT) sequence can direct the secretion of FLAG fusion proteins into the culture medium for purification using ANTI-FLAG antibodies, resins, and plates. Other vectors and expression systems are well known in the art for use with a variety of host cells.

The present invention also relates to a host cell transformed with a polynucleotide vector construct of the present invention. The host cell can be either prokaryotic or eukaryotic. Bacterial cells may be preferred prokaryotic host cells in some circumstances and typically are a strain of E. coli such as, for example, the E. coli strains DH5 available from Bethesda Research Laboratories Inc., Bethesda, Md., USA, and RR1 available from the American Type Culture Collection (ATCC) of Rockville, Md., USA (No ATCC 31343). Preferred eukaryotic host cells include yeast, insect and mammalian cells, preferably vertebrate cells such as those from a mouse, rat, monkey or human fibroblastic and colon cell lines. Yeast host cells include YPH499, YPH500 and YPH501, which are generally available from Stratagene Cloning Systems, La Jolla, Calif. 92037, USA. Preferred mammalian host cells include Chinese hamster ovary (CHO) cells available from the ATCC as CCL61, NIH Swiss mouse embryo cells NIH/3T3 available from the ATCC as CRL 1658, monkey kidney-derived COS-1 cells available from the ATCC as CRL 1650 and 293 cells which are human embryonic kidney cells. Preferred insect cells are Sf9 cells which can be transfected with baculovirus expression vectors. An overview regarding the choice of suitable host cells for expression can be found in, for example, the textbook of Paulina Balbás and Argelia Lorence “Methods in Molecular Biology Recombinant Gene Expression, Reviews and Protocols,” Part One, Second Edition, ISBN 978-1-58829-262-9, and other literature known to the person of skill.

Transformation of appropriate cell hosts with a DNA construct of the present invention is accomplished by well known methods that typically depend on the type of vector used. With regard to transformation of prokaryotic host cells, see, for example, Cohen et al (1972) Proc. Natl. Acad. Sci. USA 69, 2110, and Sambrook et al (1989) Molecular Cloning, A Laboratory Manual, Cold Spring Harbor Laboratory, Cold Spring Harbor, N.Y. Transformation of yeast cells is described in Sherman et al (1986) Methods In Yeast Genetics, A Laboratory Manual, Cold Spring Harbor, N.Y. The method of Beggs (1978) Nature 275, 104-109 is also useful. With regard to vertebrate cells, reagents useful in transfecting such cells, for example calcium phosphate and DEAE-dextran or liposome formulations, are available from Stratagene Cloning Systems, or Life Technologies Inc., Gaithersburg, Md. 20877, USA. Electroporation is also useful for transforming and/or transfecting cells and is well known in the art for transforming yeast cell, bacterial cells, insect cells and vertebrate cells.

Successfully transformed cells, i.e. cells that contain a DNA construct of the present invention, can be identified by well known techniques such as PCR. Alternatively, the presence of the protein in the supernatant can be detected using antibodies.

It will be appreciated that certain host cells of the invention are useful in the preparation of the peptides of the invention, for example bacterial, yeast and insect cells. However, other host cells may be useful in certain therapeutic methods. For example, antigen-presenting cells, such as dendritic cells, may usefully be used to express the peptides of the invention such that they may be loaded into appropriate MHC molecules. Thus, the current invention provides a host cell comprising a nucleic acid or an expression vector according to the invention.

In a preferred embodiment the host cell is an antigen presenting cell, in particular a dendritic cell or antigen presenting cell. APCs loaded with a recombinant fusion protein containing prostatic acid phosphatase (PAP) are currently under investigation for the treatment of prostate cancer (“Sipuleucel-T”) (Small et al., 2006; Rini et al., 2006; Small et al., 2006).

A further aspect of the invention provides a method of producing a peptide or its variant, the method comprising culturing a host cell and isolating the peptide from the host cell or its culture medium. A further aspect of the invention relates to a method of producing a peptide according to the present invention, the method comprising culturing the host cell according to the present invention, expressing the nucleic acid or the expression vector according to the present invention, and isolating the peptide from said host cell or its culture medium, as described herein and in the respective literature.

Yet another aspect of the present invention then relates to the AVL polypeptide according to the present invention, the peptide according to the present invention, the nucleic acid or expression vector according to the present invention, the pharmaceutical composition according to the present invention, the antibody according to the present invention, the CTL according to the present invention, or the host cell according to the present invention for use in medicine. For example, the peptide or its variant may be prepared for intravenous (i.v.) injection, subcutaneous (s.c.) injection, intradermal (i.d.) injection, intraperitoneal (i.p.) injection, intramuscular (i.m.) injection. Preferred methods of peptide injection include s.c., i.d., i.p., i.m., and i.v. Preferred methods of DNA injection include i.d., i.m., s.c., i.p. and i.v. Doses of e.g. between 50 μg and 1.5 mg, preferably 125 μg to 500 μg, of peptide or DNA may be given and will depend on the respective peptide or DNA. Doses of this range were successfully used in previous trials (Brunsvig et al., 2006; Staehler et al., 2007).

Another aspect of the present invention includes an in vitro method for producing activated T cells, the method comprising contacting in vitro T cells with antigen loaded human class I or II MHC molecules expressed on the surface of a suitable antigen-presenting cell for a period of time sufficient to activate the T cell in an antigen specific manner, wherein the antigen is a peptide according to the invention. Preferably a sufficient amount of the antigen is used with an antigen-presenting cell.

In the case of a MHC class II epitope being used as an antigen, the T cells are CD4-positive helper cells, preferably of T_{H1}-type. The MHC class II molecules may be expressed on the surface of any suitable cell. Preferably the cell does not naturally express MHC class II molecules (in which case the cell has been transfected in order to express such a molecule). Alternatively, if the cell naturally expresses MHC class II molecules, it is preferred that it is defective in the antigen-processing or antigen-presenting pathways. In this way, it is possible for the cell expressing the MHC class II molecule to be completely loaded with a chosen peptide antigen before activating the T cell.

The antigen-presenting cell (or stimulator cell) typically has MHC class II molecules on its surface and preferably is itself substantially incapable of loading said MHC class II molecule with the selected antigen. The MHC class II molecule may readily be loaded with the selected antigen in vitro.

Preferably the mammalian cell lacks or has a reduced level or function of the TAP peptide transporter. Suitable cells that lack the TAP peptide transporter include T2, RMA-S and Drosophila cells. TAP is the Transporter associated with Antigen Processing.

The human peptide loading deficient cell line T2 is available from the American Type Culture Collection, 12301 Parklawn Drive, Rockville, Md. 20852, USA under Catalogue No CRL 1992; the Drosophila cell line Schneider line 2 is available from the ATCC under Catalogue No CRL 19863; the mouse RMA-S cell line is described in (Ljunggren and Karre, 1985). Preferably, the host cell before transfection expresses substantially no MHC class 1 molecules.

The present invention further relates to a particular marker protein that can be used in the diagnosis and prognosis of gastric cancer. Therefore, a particular aspect of the present invention provides the identity of a protein that is up-regulated in aggressive gastric cancer. As provided herein, the protein AVL9, is shown to play an important role in tissue remodeling required for tumor growth in the nervous system. Therefore the expression of AVL9 (e.g., human AVL9 having the amino acid sequence of SEQ ID NO: 7, encoded by the nucleic acid sequence of SEQ ID NO: 6 in cells obtained from the stomach or other tumorous specimen) can be used as a marker to distinguish gastric cancer from other forms of cancer.

Therefore, another aspect of the present invention relates to a method for diagnosing cancer, comprising detecting the presence of at least one peptide derived from the protein AVL9 presented on the surface of a cell and/or the level of expression of the gene AVL9 in a biological sample obtained from a mammal, wherein the presence of said peptide or an increase of the level of expression of the gene AVL9 in said sample compared to a biological non-cancer sample is indicative for cancer. Preferably, the mammal is a human.

Preferred is the method according to the present invention, wherein said proliferative disorder is selected from benign prostatic hyperplasia, gastric cancer, NSCLC, renal cell carcinoma, glioblastoma or colorectal carcinoma.

Therefore, the present invention provides methods of identifying a mammal, preferably a human that is likely to have gastric cancer. In one embodiment, the likelihood determined is between 80% and 100%. One such method comprises determining the level of AVL9 in a tumor sample from the mammalian subject. In one embodiment, the sample is obtained by radical surgery. In another embodiment, the sample is obtained by needle biopsy.

When the level of AVL9 determined is 20% or more up-regulated in cells relative to that determined in benign epithelial cells of the same specimen, the mammalian subject is identified as being likely to have gastric cancer. In one embodiment the determination of the level of AVL9 is performed in situ. In another embodiment the determination of the level of AVL9 is performed in vitro. In still another embodiment, the determination of the level of AVL9 is performed in vivo. In a preferred embodiment, the determination of the level of AVL9 is performed by Laser Capture Microscopy coupled with a Western blot.

In a particularly preferred method according to the present invention, the determination of the level of AVL9 is performed with an antibody specific for AVL9, i.e. said detecting comprises the use of an antibody which specifically recognizes the AVL9 polypeptide.

In a particularly preferred method according to the present invention, the determination of the level of AVL9 is performed with a fusion peptide comprising an AVL9-derived sequence, or a nucleic acid hybridizing under stringent conditions to the nucleic acid according to SEQ ID NO: 6. In another such embodiment the determination of the level of AVL9 is performed by PCR with a primer specific for an mRNA encoding AVL9. In still another embodiment the determination of the level of AVL9 is performed with a nucleotide probe specific for an mRNA encoding AVL9. In one such embodiment, the determination of the level of AVL9 is performed by a Northern blot. In another embodiment, the determination of the level of AVL9 is performed by a ribonuclease protection assay. In other embodiments, immunological tests such as enzyme-linked immunosorbent assays (ELISA), radioimmunoassays (RIA), and Western blots may be used to detect AVL9 polypeptides in a body fluid sample (such as blood, serum, sputum, urine, or peritoneal fluid). Biopsies, tissue samples, and cell samples (such as ovaries, lymph nodes, ovarian surface epithelial cell scrapings, lung biopsies, liver biopsies, and any fluid sample containing cells (such as peritoneal fluid, sputum, and pleural effusions) may be tested by disaggregating and/or solubilizing the tissue or cell sample and subjecting it to an immunoassay for polypeptide detection, such as ELISA, RIA, or Western blotting. Such cell or tissue samples may also be analyzed by nucleic acid-based methods, e.g., reverse transcription-polymerase chain reaction (RT-PCR) amplification, Northern hybridization, or slot- or dot-blotting.

In order to visualize the distribution of tumor cells within a tissue sample, diagnostic tests that preserve the tissue structure of a sample, e.g., immunohistological staining, in situ RNA hybridization, or in situ RT-PCR may be employed to detect gastric cancer marker polypeptide or mRNA, respectively. For in vivo localization of tumor masses, imaging tests such as magnetic resonance imaging (MRI) may be employed by introducing into the subject an antibody that specifically binds a AVL9 polypeptide (particularly a cell surface-localized polypeptide), wherein the antibody is conjugated or otherwise coupled to a paramagnetic tracer (or other appropriate detectable moiety, depending upon the imaging method used); alternatively, localization of an unlabeled tumor marker-specific antibody may be detected using a secondary antibody coupled to a detectable moiety.

Antibodies to the AVL9 polypeptides, to the chimeric/fusion proteins comprising the AVL9 polypeptides, as well as to the fragments of the AVL9 polypeptides, including proteolytic, and antigenic fragments, and to the chimeric/fusion proteins/peptides comprising these fragments are also part of the present invention. In addition, methods of using such antibodies for the prognosis of cancer, and gastric cancer in particular, are also part of the present invention. The antibodies of the present invention can be polyclonal antibodies, monoclonal antibodies and/or chimeric antibodies. Immortal cell lines that produce a monoclonal antibody of the present invention are also part of the present invention.

One of ordinary skill in the art will understand that in some instances, higher expression of AVL9 as a tumor marker gene will indicate a worse prognosis for a subject having gastric cancer. For example, relatively higher levels AVL9 expression may indicate a relative large primary tumor, a higher tumor burden (e.g., more metastases), or a relatively more malignant tumor phenotype. The diagnostic and prognostic methods of the invention involve using known methods, e.g., antibody-based methods to detect AVL9 polypeptides and nucleic acid hybridization- and/or amplification-based methods to detect AVL9 mRNA as described above.

In addition, since rapid tumor cell destruction often results in autoantibody generation, the gastric cancer tumor markers of the invention may be used in serological assays (e.g., an ELISA test of a subject's serum) to detect auto-antibodies against AVL9 in a subject. AVL9 polypeptide-specific autoantibody levels that are at least about 3-fold higher (and preferably at least 5-fold or 7-fold higher, most preferably at least 10-fold or 20-fold higher) than in a control sample are indicative of gastric cancer.

Cell-surface localized, intracellular, and secreted AVL9 polypeptides may all be employed for analysis of biopsies, e.g., tissue or cell samples (including cells obtained from liquid samples such as peritoneal cavity fluid) to identify a tissue or cell biopsy as containing gastric cancer cells. A biopsy may be analyzed as an intact tissue or as a whole-cell sample, or the tissue or cell sample may be disaggregated and/or solubilized as necessary for the particular type of diagnostic test to be used. For example, biopsies or samples may be subjected to whole-tissue or whole-cell analysis of AVL9 polypeptide or mRNA levels in situ, e.g., using immunohistochemistry, in situ mRNA hybridization, or in situ RT-PCR. The skilled artisan will know how to process tissues or cells for analysis of polypeptide or mRNA levels using immunological methods such as ELISA, immunoblotting, or equivalent methods, or analysis of mRNA levels by nucleic acid-based analytical methods such as RT-PCR, Northern hybridization, or slot- or dot-blotting.

Another aspect of the present invention then relates to a diagnostic or therapeutic kit comprising a) a container containing a pharmaceutical composition according to the present invention as described herein in solution or in lyophilized form; b) optionally, a second container containing a diluent or reconstituting solution for the lyophilized formulation; c) optionally, at least one peptide selected from the group consisting of the peptides according to SEQ ID NOs 8 to 47; d) optionally, primary and secondary antibodies, and suitable detection reagents, such as detectable moieties, enzyme substrates, and color reagents; and d) optionally, instructions for (i) use of the solution or (ii) reconstitution and/or use of the lyophilized formulation. Preferably, said kit according to the present invention can further comprise one or more of (iii) a buffer, (iv) a diluent, (v) a filter, (vi) a needle, or (v) a syringe. The container is preferably a bottle, a vial, a syringe or test tube; and it may be a multi-use container. The pharmaceutical composition is preferably lyophilized.

Kits of the present invention preferably comprise a lyophilized formulation of the present invention in a suitable container and instructions for its reconstitution and/or use. Suitable containers include, for example, bottles, vials (e.g. dual chamber vials), syringes (such as dual chamber syringes) and test tubes. The container may be formed from a variety of materials such as glass or plastic. Preferably the kit and/or container contain/s instructions on or associated with the container that indicates directions for reconstitution and/or use. For example, the label may indicate that the lyophilized formulation is to be reconstituted to peptide concentrations as described above. The label may further indicate that the formulation is useful or intended for subcutaneous administration.

The container holding the formulation may be a multi-use vial, which allows for repeat administrations (e.g., from 2-6 administrations) of the reconstituted formulation. The kit may further comprise a second container comprising a suitable diluent (e.g., sodium bicarbonate solution).

Upon mixing of the diluent and the lyophilized formulation, the final peptide concentration in the reconstituted formulation is preferably at least 0.15 mg/ml/peptide (=75 μg) and preferably not more than 3 mg/ml/peptide (=1500 μg). The kit may further include other materials desirable from a commercial and user standpoint, including other buffers, diluents, filters, needles, syringes, and package inserts with instructions for use.

Kits of the present invention may have a single container that contains the formulation of the pharmaceutical compositions according to the present invention with or without other components (e.g., other compounds or pharmaceutical compositions of these other compounds) or may have distinct container for each component.

Preferably, kits of the invention include a formulation of the invention packaged for use in combination with the co-administration of a second compound (such as adjuvants (e.g. GM-CSF), a chemotherapeutic agent, a natural product, a hormone or antagonist, a anti-angiogenesis agent or inhibitor, a apoptosis-inducing agent or a chelator) or a pharmaceutical composition thereof. The components of the kit may be pre-complexed or each component may be in a separate distinct container prior to administration to a patient. The components of the kit may be provided in one or more liquid solutions, preferably, an aqueous solution, more preferably, a sterile aqueous solution. The components of the kit may also be provided as solids, which may be converted into liquids by addition of suitable solvents, which are preferably provided in another distinct container.

Usually, when there is more than one component, the kit will contain a second vial or other container, which allows for separate dosing. The kit may also contain another container for a pharmaceutically acceptable liquid. Preferably, a therapeutic kit will contain an apparatus (e.g., one or more needles, syringes, eye droppers, pipette, etc.), which enables administration of the agents of the invention that are components of the present kit.

Particularly preferred is a diagnostic kit according to the present invention, comprising components for detecting expression levels of AVL9 as a gastric cancer marker gene, such as, for example, a control antibody which specifically binds to a gastric marker polypeptide, such as AVL9, one or more nucleic acids which under stringent conditions hybridize to AVL9 mRNA, and, optionally, a control, such as, for example, a given amount of a particular gastric cancer marker gene polypeptide, such as AVL9. A kit for detecting gastric cancer marker mRNA preferably contains one or more nucleic acids (e.g., one or more oligonucleotide primers or probes, DNA probes, RNA probes, or templates for generating RNA probes) that specifically hybridize with AVL9 mRNA.

Particularly, the antibody-based kit can be used to detect the presence of, and/or measure the level of, a AVL9 polypeptide that is specifically bound by the antibody or an immunoreactive fragment thereof. The kit can include an antibody reactive with the antigen and a reagent for detecting a reaction of the antibody with the antigen. Such a kit can be an ELISA kit and can contain a control (e.g., a specified amount of a particular gastric cancer marker polypeptide), primary and secondary antibodies when appropriate, and any other necessary reagents such as detectable moieties, enzyme substrates and color reagents as described above. The diagnostic kit can, alternatively, be an immunoblot kit generally comprising the components and reagents described herein.

Antibodies for diagnostic use may be labeled with probes suitable for detection by various imaging methods. Methods for detection of probes include, but are not limited to, fluorescence, light, confocal and electron microscopy; magnetic resonance imaging and spectroscopy; fluoroscopy, computed tomography and positron emission tomography. Suitable probes include, but are not limited to, fluorescein, rhodamine, eosin and other fluorophores, radioisotopes, gold, gadolinium and other lanthanides, paramagnetic iron, fluorine-18 and other positron-emitting radionuclides. Additionally, probes may be bi- or multi-functional and be detectable by more than one of the methods listed. These antibodies may be directly or indirectly labeled with said probes. Attachment of probes to the antibodies includes covalent attachment of the probe, incorporation of the probe into the antibody, and the covalent attachment of a chelating compound for binding of probe, amongst others well recognized in the art. For immunohistochemistry, the disease tissue sample may be fresh or frozen or may be embedded in paraffin and fixed with a preservative such as formalin. The fixed or embedded section contains the sample are contacted with a labeled primary antibody and secondary antibody, wherein the antibody is used to detect the AVL9 protein express in situ.

A nucleic acid-based kit can be used to detect and/or measure the expression level of AVL9 by detecting and/or measuring the amount of AVL9 mRNA in a sample, such as a tissue or cell biopsy. For example, an RT-PCR kit for detection of elevated expression of AVL9 preferably contains oligonucleotide primers sufficient to perform reverse transcription of gastric cancer marker mRNA to cDNA and PCR amplification of gastric cancer marker cDNA, and will preferably also contain control PCR template molecules and primers to perform appropriate negative and positive controls, and internal controls for quantization. One of ordinary skill in the art will understand how to select the appropriate primers to perform the reverse transcription and PCR reactions, and the appropriate control reactions to be performed. Such guidance is found, for example, in F. Ausubel et al., Current Protocols in Molecular Biology, John Wiley & Sons, New York, N.Y., 1997. Numerous variations of RT-PCR are known in the art.

Yet another aspect of the present invention then relates to a method for producing activated cytotoxic T lymphocytes (CTL) and/or T helper cells, wherein the method comprises contacting CTL in vitro with antigen loaded human class I or II MHC molecules expressed on the surface of a suitable antigen-presenting cell or an artificial construct mimicking an antigen-presenting cell (see, for example Turtle C J, Riddell S R. Artificial antigen-presenting cells for use in adoptive immunotherapy. Cancer J. 2010 July-August; 16(4):374-81) for a period of time sufficient to activate said CTL in an antigen specific manner, wherein said antigen is a peptide according to the present invention.

Yet another aspect of the present invention then relates to the AVL polypeptide according to the present invention, the peptide according to the present invention, the nucleic acid or expression vector according to the present invention, the pharmaceutical composition according to the present invention, the antibody according to the present invention, the CTL according to the present invention, or the host cell according to the present invention for the treatment of proliferative disorders such as cancer, gastric cancer, NSCLC, renal cell carcinoma, benign prostatic hyperplasia or colorectal carcinoma. Thus, the present invention further relates to the use of the AVL polypeptide as a novel target for cancer treatment. Methods of treating cancer cells and gastric cancer cells are also provided.

Yet another aspect of the present invention then relates to a method for killing target cells in a patient which target cells aberrantly express a polypeptide comprising an amino acid sequence as given in any of SEQ ID NOs 1 to 5, wherein the method comprises administering to said patient an effective amount of cytotoxic T lymphocytes (CTL) as produced according to the present invention.

Any molecule of the invention, i.e. the peptide, nucleic acid, expression vector, cell, activated CTL, T-cell receptor or the nucleic acid encoding it is useful for the treatment of disorders as described herein, in particular gastric cancer, characterized by cells escaping an immune response. Therefore any molecule of the present invention may be used as medicament or in the manufacture of a medicament. The molecule may be used by itself or combined with other molecule(s) of the invention or (a) known molecule(s).

The present invention provides a method for treating or monitoring cancer in a patient, comprising a method for diagnosis according to the present invention as described above, and treating said cancer in said patient based on said diagnostic result. The cancer is selected from, in particular, gastric cancer, renal cell carcinoma, colon cancer, non-small cell lung carcinoma, adenocarcinoma, prostate cancer, and malignant melanoma.

The peptides according to the invention can be used to generate and develop specific antibodies against MHC/peptide complexes. These can be used for therapy, targeting toxins or radioactive substances to the diseased tissue. Another use of these antibodies can be targeting radionuclides to the diseased tissue for imaging purposes such as PET. This use can help to detect small metastases or to determine the size and precise localization of diseased tissues.

Targeted Delivery of immunotoxins to AVL9 can be employed as therapeutic targets for the treatment or prevention of gastric cancer. For example, an antibody molecule that specifically binds a cell surface-localized AVL9 polypeptide can be conjugated to a radioisotope or other toxic compound. Antibody conjugates are administered to the subject so that the binding of the antibody to its cognate gastric cancer polypeptide results in the targeted delivery of the therapeutic compound to gastric cancer cells, thereby treating an ovarian cancer.

The therapeutic moiety can be a toxin, radioisotope, drug, chemical, or a protein (see, e.g., Bera et al. “Pharmacokinetics and antitumor activity of a bivalent disulfide-stabilized Fv immunotoxin with improved antigen binding to erbB2” Cancer Res. 59:4018-4022 (1999)). For example, the antibody can be linked or conjugated to a bacterial toxin (e.g., diphtheria toxin, pseudomonas exotoxin A, cholera toxin) or plant toxin (e.g., ricin toxin) for targeted delivery of the toxin to a cell expressing AVL9. This immunotoxin can be delivered to a cell and upon binding the cell surface-localized gastric cancer marker polypeptide, the toxin conjugated to the gastric cancer marker-specific antibody will be delivered to the cell.

In addition, for any AVL9 polypeptide for which there is a specific ligand (e.g., a ligand that binds a cell surface-localized protein), the ligand can be used in place of an antibody to target a toxic compound to a gastric cancer cell, as described above.

Because the TUMAPs according to SEQ IDs 1 to 5 of the invention as derived from the gastric cancer tumor marker AVL9 are overpresented in gastric cancer cells and are not or only at extremely low levels presented in normal cells, inhibition of AVL9 expression or polypeptide activity may be integrated into any therapeutic strategy for treating or preventing gastric cancer.

The principle of antisense therapy is based on the hypothesis that sequence-specific suppression of gene expression (via transcription or translation) may be achieved by intra-cellular hybridization between genomic DNA or mRNA and a complementary antisense species. The formation of such a hybrid nucleic acid duplex interferes with transcription of the target tumor antigen-encoding genomic DNA, or processing/transport/translation and/or stability of the target tumor antigen mRNA.

Antisense nucleic acids can be delivered by a variety of approaches. For example, antisense oligonucleotides or anti-sense RNA can be directly administered (e.g., by intravenous injection) to a subject in a form that allows uptake into tumor cells. Alternatively, viral or plasmid vectors that encode antisense RNA (or RNA fragments) can be introduced into cells in vivo. Antisense effects can also be induced by sense sequences; however, the extent of phenotypic changes is highly variable. Phenotypic changes induced by effective antisense therapy are assessed according to changes in, e.g., target mRNA levels, target protein levels, and/or target protein activity levels.

In a specific example, inhibition of gastric cancer marker function by antisense gene therapy may be accomplished by direct administration of antisense gastric cancer marker RNA to a subject. The antisense tumor marker RNA may be produced and isolated by any standard technique, but is most readily produced by in vitro transcription using an antisense tumor marker cDNA under the control of a high efficiency promoter (e.g., the T7 promoter). Administration of anti-sense tumor marker RNA to cells can be carried out by any of the methods for direct nucleic acid administration described below.

An alternative strategy for inhibiting AVL9 function using gene therapy involves intracellular expression of an anti-AVL9 antibody or a portion of an anti-AVL9 antibody. For example, the gene (or gene fragment) encoding a monoclonal antibody that specifically binds to a AVL9 polypeptide and inhibits its biological activity is placed under the transcriptional control of a specific (e.g., tissue- or tumor-specific) gene regulatory sequence, within a nucleic acid expression vector. The vector is then administered to the subject such that it is taken up by gastric cancer cells or other cells, which then secrete the anti-AVL9 antibody and thereby block biological activity of the AVL9 polypeptide. Preferably, the AVL9 polypeptide is present at the extracellular surface of gastric cancer cells.

In the methods described above, which include the administration and uptake of exogenous DNA into the cells of a subject (i.e., gene transduction or transfection), the nucleic acids of the present invention can be in the form of naked DNA or the nucleic acids can be in a vector for delivering the nucleic acids to the cells for inhibition of gastric cancer marker protein expression. The vector can be a commercially available preparation, such as an adenovirus vector (Quantum Biotechnologies, Inc. (Laval, Quebec, Canada). Delivery of the nucleic acid or vector to cells can be via a variety of mechanisms. As one example, delivery can be via a liposome, using commercially available liposome preparations such as LIPOFECTIN, LIPOFECTAMINE (GIBCO-25 BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the nucleic acid or vector of this invention can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

As one example, vector delivery can be via a viral system, such as a retroviral vector system that can package a recombinant retroviral genome. The recombinant retrovirus can then be used to infect and thereby deliver to the infected cells antisense nucleic acid that inhibits expression of AVL9. The exact method of introducing the altered nucleic acid into mammalian cells is, of course, not limited to the use of retroviral vectors. Other techniques are widely available for this procedure including the use of adenoviral vectors, adeno-associated viral (AAV) vectors, lentiviral vectors, pseudotyped retroviral vectors. Physical transduction techniques can also be used, such as liposome delivery and receptor-mediated and other endocytosis mechanisms. This invention can be used in conjunction with any of these or other commonly used gene transfer methods.

Since the peptides of the invention derived from AVL9 were isolated from gastric cancer, the pharmaceutical formulation of the invention is preferably used to treat gastric cancer.

The present invention will now be described in the following examples that describe preferred embodiments thereof, nevertheless, without being limited thereto. For the purposes of the present invention, all references as cited herein are incorporated by reference in their entireties.

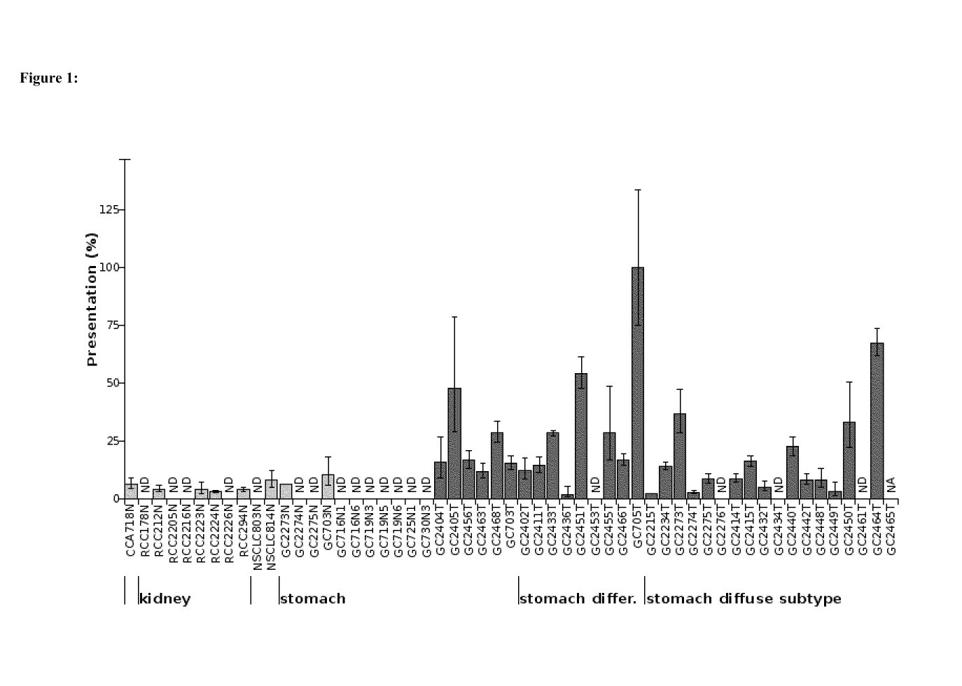

FIG. 1 shows a quantitative peptide presentation plot illustrating the average presentation for a peptide in distinct samples visualized in a bar chart. The presentation is expressed in percent as abundance relative to the maximum area. The variation is visualized as 95% confidence intervals based on the measured replicates. If the peptide was identified in a sample but no quantification was possible, it is indicated by the label NA (not available/no area). The reason can be either a problem in the Feature finding of the LCMS run or during the normalization of the sample. Sample without detection of this peptide are marked as ND. All normal tissue samples and all samples of gastric cancer investigated are shown provided that they meet appropriate quality control criteria.

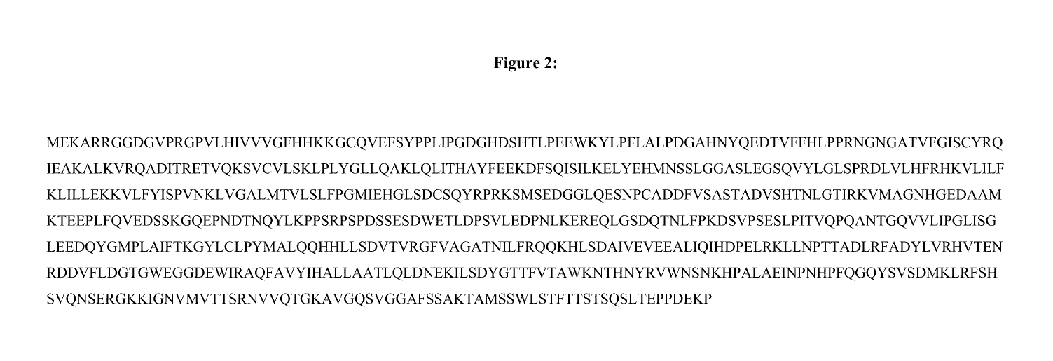

FIG. 2 shows the amino acid sequence of the protein AVL9.

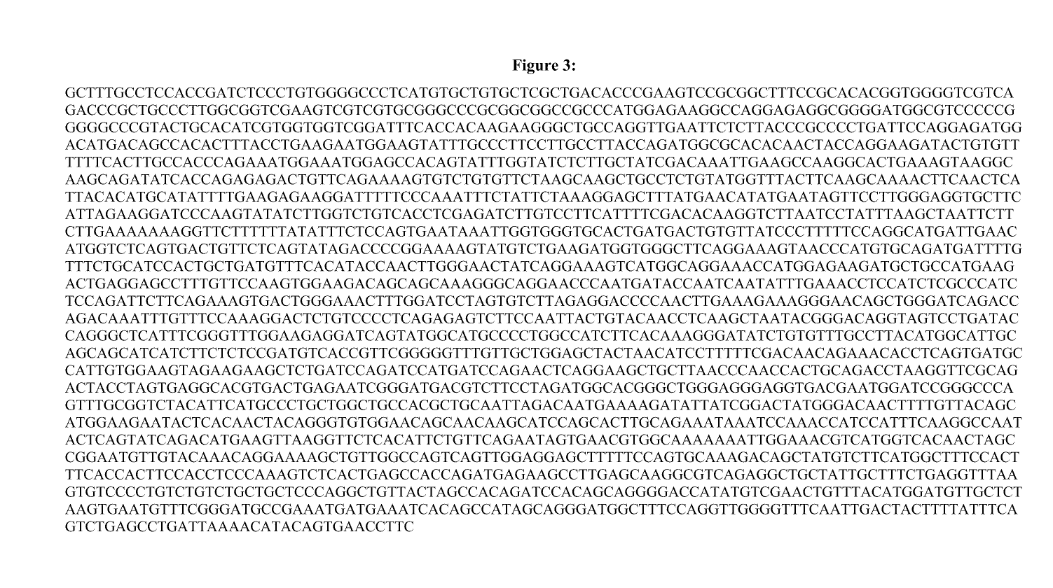

FIG. 3 shows the mRNA sequence of AVL9.

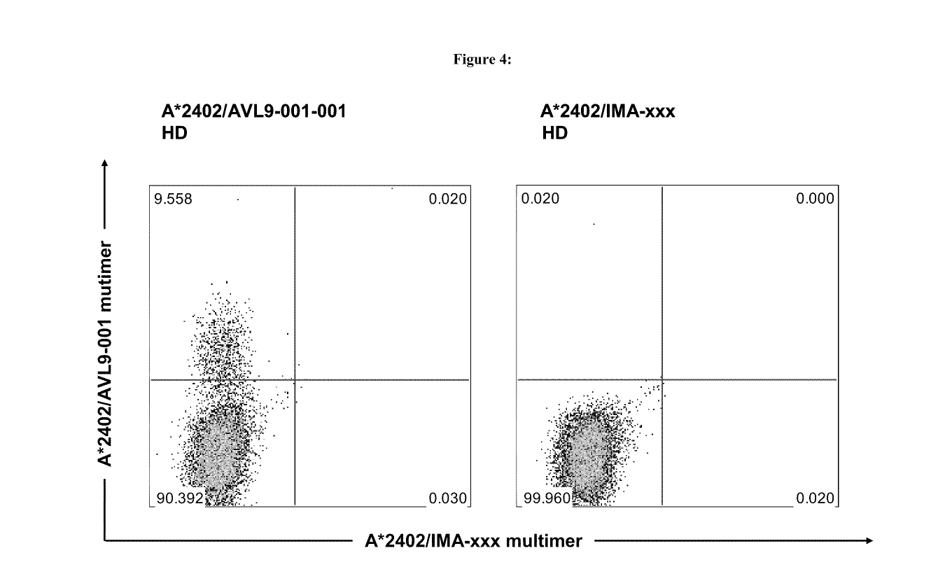

FIG. 4 shows exemplary results of peptide-specific in vitro CD8+ T-cell responses of a healthy HLA-A*24+ donor determined by flow cytometric analysis for one peptide of the invention. CD8+ T cells were primed using artificial antigen presenting cells loaded with AVL9-001 (left panel) or irrelevant peptide IMA-xxx (right panel), respectively. After three cycles of stimulation, the detection of peptide-reactive cells was performed by double staining with AVL9-001-plus IMA-xxx A*2402-multimers. Shown cells were gated on CD8+ lymphocytes.

SEQ ID No 1 to SEQ ID No 5 show the amino acid sequences of the peptides of the invention.

SEQ ID No 6 shows the amino acid sequence of the AVL9 polypeptide.

SEQ ID No 7 shows the nucleic acid sequence encoding the AVL9 polypeptide according to SEQ ID No 6.

SEQ ID No 8 to SEQ ID No 47 show additional peptides as used in the preparations of the present invention.

### EXAMPLES

### Example 1

### Identification of Tumor Associated Peptides Presented on Cell Surface

Tissue Samples

Patients' tumor tissues were provided by Kyoto Prefectural University of Medicine (KPUM), Kyoto, Japan, and Osaka City University Graduate School of Medicine (OCU), Osaka, Japan. Written informed consents of all patients had been given before surgery. Tissues were shock-frozen in liquid nitrogen immediately after surgery and stored until isolation of TUMAPs at −80° C.

Isolation of HLA Peptides from Tissue Samples

HLA peptide pools from shock-frozen tissue samples were obtained by immune precipitation from solid tissues according to a slightly modified protocol (Falk et al., 1991) (Seeger et al., 1999) using the HLA-A, -B, -C-specific antibody W6/32, CNBr-activated sepharose, acid treatment, and ultrafiltration.

### Sequence Identification

The HLA peptide pools as obtained were separated according to their hydrophobicity by reversed-phase chromatography (Acquity HPLC system, Waters) and the eluting peptides were analyzed in an LTQ-Orbitrap hybrid mass spectrometer (ThermoElectron) equipped with an ESI source. Peptide pools were loaded directly onto the analytical fused-silica micro-capillary column (75 μm i.d.×250 mm) packed with 1.7 μm C18 reversed-phase material (Waters) applying a flow rate of 400 nl per minute. Subsequently, the peptides were separated using a two-step 180 minute-binary gradient from 10% to 33% B at a flow rate of 300 nl per minute. The gradient was composed of Solvent A (0.1% formic acid in water) and solvent B (0.1% formic acid in acetonitrile). A gold coated glass capillary (PicoTip, New Objective) was used for introduction into the nanoESI source. The LTQ-Orbitrap mass spectrometer was operated in the data-dependent mode using a TOP5 strategy. In brief, a scan cycle was initiated with a full scan of high mass accuracy in the orbitrap (R=30 000), which was followed by MS/MS scans also in the orbitrap (R=7500) on the 5 most abundant precursor ions with dynamic exclusion of previously selected ions.

Tandem mass spectra were interpreted by SEQUEST and additional manual control. The identified peptide sequence was assured by comparison of the generated natural peptide fragmentation pattern with the fragmentation pattern of a synthetic sequence-identical reference peptide.

Relative TUMAP Quantification

LCMS survey data was analyzed independently of the Tandem-MS making use of the high-mass accuracy. To extract LCMS signals as well as the signal areas (ion counting) the program SuperHirn (Mueller et al., 2007) was used. Thus each identified peptide can be associated with quantitative data allowing relative quantification between samples and tissues. To account for variation between technical and biological replicates, a two-tier normalization scheme was used based on central tendency normalization. The normalization assumes that most measured signals result from house-keeping peptides and the small fraction of over-presented peptides does not influence the central tendency of the data significantly. In the first normalization step the replicates of the same sample are normalized by calculating the mean presentation for each peptide in the respective replicate set. This mean is used to compute normalization factors for each peptide and LC-MS run. Averaging over all peptides results in run-wise normalization factors which are applied to all peptides of the particular LCMS run. This approach ensures that systematic intra-sample variation is removed, e.g. due to different injection volumes between the replicate runs.

Only peptides which have a coefficient of variation smaller than 25% between their replicate areas are considered in the next normalization step. Again the mean presentation of each peptide is calculated, this time for all samples of a defined preparation antibody (e.g. W6/32). The mean is used to compute normalization factors for each peptide and sample. Averaging over all peptides results in sample-wise normalization factors which are applied to all peptides of the particular sample. Systematic bias due to different tissue weights or MHC expression levels is therefore removed.

Combining LCMS-survey and Tandem-MS data sets yielded quantitative data for each identified peptide. To identify over-presented peptides, a presentation profile (FIG. 1) was calculated showing the median presentation of the peptide in each sample as well as replicate variation. The profile juxtaposes samples of the tumor entity of interest to a baseline of normal tissue samples. Each of these profiles was consolidated into an over-presentation score by calculating the p-value of a Linear Mixed-Effects Model (Pinheiro et al., 2008) (GNU R) adjusting for multiple testing by False Discovery Rate (Benjamini and Hochberg, 1995).

### Example 2

### Expression Profiling of Genes Encoding the Peptides of the Invention

Not all peptides identified as being presented on the surface of tumor cells by MHC molecules are suitable for immunotherapy, because the majority of these peptides are derived from normal cellular proteins expressed by many cell types. Only few of these peptides are tumor-associated and likely able to induce T cells with a high specificity of recognition for the tumor from which they were derived. In order to identify such peptides and minimize the risk for autoimmunity induced by vaccination the inventors focused on those peptides that are derived from proteins that are over-expressed on tumor cells compared to the majority of normal tissues.

The ideal peptide will be derived from a protein that is unique to the tumor and not present in any other tissue. To identify peptides that are derived from genes with an expression profile similar to the ideal one the identified peptides were assigned to the proteins and genes, respectively, from which they were derived and expression profiles of these genes were generated.

RNA Sources and Preparation

Surgically removed tissue specimens were provided by two different clinical sites (see Example 1) after written informed consent had been obtained from each patient. Tumor tissue specimens were snap-frozen in liquid nitrogen immediately after surgery and later homogenized with mortar and pestle under liquid nitrogen. Total RNA was prepared from these samples using TRI Reagent (Ambion, Darmstadt, Germany) followed by a cleanup with RNeasy (QIAGEN, Hilden, Germany); both methods were performed according to the manufacturer's protocol.

Total RNA from healthy human tissues was obtained commercially (Ambion, Huntingdon, UK; Clontech, Heidelberg, Germany; Stratagene, Amsterdam, Netherlands; BioChain, Hayward, Calif., USA). The RNA from several individuals (between 2 and 123 individuals) was mixed such that RNA from each individual was equally weighted. Leukocytes were isolated from blood samples of four healthy volunteers.

Quality and quantity of all RNA samples were assessed on an Agilent 2100 Bioanalyzer (Agilent, Waldbronn, Germany) using the RNA 6000 Pico LabChip Kit (Agilent).

Microarray Experiments

Gene expression analysis of all tumor and normal tissue RNA samples was performed by Affymetrix Human Genome (HG) U133A or HG-U133 Plus 2.0 oligonucleotide microarrays (Affymetrix, Santa Clara, Calif., USA). All steps were carried out according to the Affymetrix manual. Briefly, double-stranded cDNA was synthesized from 5-8 μg of total RNA, using SuperScript RTII (Invitrogen) and the oligo-dT-T7 primer (MWG Biotech, Ebersberg, Germany) as described in the manual. In vitro transcription was performed with the BioArray High Yield RNA Transcript Labelling Kit (ENZO Diagnostics, Inc., Farmingdale, N.Y., USA) for the U133A arrays or with the GeneChip IVT Labelling Kit (Affymetrix) for the U133 Plus 2.0 arrays, followed by cRNA fragmentation, hybridization, and staining with streptavidin-phycoerythrin and biotinylated anti-streptavidin antibody (Molecular Probes, Leiden, Netherlands). Images were scanned with the Agilent 2500A GeneArray Scanner (U133A) or the Affymetrix Gene-Chip Scanner 3000 (U133 Plus 2.0), and data were analyzed with the GCOS software (Affymetrix), using default settings for all parameters. For normalization, 100 housekeeping genes provided by Affymetrix were used. Relative expression values were calculated from the signal log ratios given by the software and the normal kidney sample was arbitrarily set to 1.0.

### Example 3

### In Vitro Immunogenicity for MHC Class I Presented Peptides

To get information regarding the immunogenicity of the TUMAPs of the present invention, the inventors performed investigations using a well established in vitro stimulation platform already described by (Walter, S, Herrgen, L, Schoor, O, Jung, G, Wernet, D, Buhring, H J, Rammensee, H G, and Stevanovic, S; 2003, Cutting edge: predetermined avidity of human CD8 T cells expanded on calibrated MHC/anti-CD28-coated microspheres, J. Immunol., 171, 4974-4978). This way the inventors could show immunogenicity for 32 HLA-A*2402 restricted TUMAPs of the invention demonstrating that these peptides are T-cell epitopes against which CD8+ precursor T cells exist in humans (Table 4).

In vitro priming of CD8+ T cells In order to perform in vitro stimulations by artificial antigen presenting cells (aAPC) loaded with peptide-MHC complex (pMHC) and anti-CD28 antibody, the inventors first isolated CD8 T cells from fresh HLA-A*24 leukapheresis products of healthy donors obtained from the Blood Bank Tuebingen.

CD8 T cells were either directly enriched from the leukapheresis product or PBMCs (peripheral blood mononuclear cells) were isolated first by using standard gradient separation medium (PAA, Cölbe, Germany). Isolated CD8 lymphocytes or PBMCs were incubated until use in T-cell medium (TCM) consisting of RPMI-Glutamax (Invitrogen, Karlsruhe, Germany) supplemented with 10% heat inactivated human AB serum (PAN-Biotech, Aidenbach, Germany), 100 U/ml Penicillin/100 μg/ml Streptomycin (Cambrex, Cologne, Germany), 1 mM sodium pyruvate (CC Pro, Oberdorla, Germany), 20 μg/ml Gentamycin (Cambrex). 2.5 ng/ml IL-7 (PromoCell, Heidelberg, Germany) and 10 U/ml IL-2 (Novartis Pharma, Nürnberg, Germany) were also added to the TCM at this step. Isolation of CD8+ lymphocytes was performed by positive selection using CD8 MicroBeads (Miltenyi Biotec, Bergisch-Gladbach, Germany).

Generation of pMHC/anti-CD28 coated beads, T-cell stimulations and readout was performed as described before (Walter et al., 2003) with minor modifications. Briefly, biotinylated peptide-loaded recombinant HLA-A*2402 molecules lacking the transmembrane domain and biotinylated at the carboxy terminus of the heavy chain were produced. The purified costimulatory mouse IgG2a anti human CD28 Ab 9.3 (Jung et al., 1987) was chemically biotinylated using sulfo-N-hydroxysuccinimidobiotin as recommended by the manufacturer (Perbio, Bonn, Germany). Beads used were 5.6 μm large streptavidin coated polystyrene particles (Bangs Laboratories, Illinois, USA). pMHC used as controls were A*0201/MLA-001 (peptide ELAGIGILTV from modified Melan-A/MART-1) and A*0201/DDX5-001 (YLLPAIVHI from DDX5), respectively.

800.000 beads/200 μl were coated in 96-well plates in the presence of 600 ng biotin anti-CD28 plus 200 ng relevant biotin-pMHC (high density beads). Stimulations were initiated in 96-well plates by co-incubating 1×10⁶ CD8+ T cells with 2×10⁵ washed coated beads in 200 μl TCM supplemented with 5 ng/ml IL-12 (PromoCell) for 3-4 days at 37° C. Half of the medium was then exchanged by fresh TCM supplemented with 80 U/ml IL-2 and incubating was continued for 3-4 days at 37° C. This stimulation cycle was performed for a total of three times. Finally, multimeric analyses were performed by staining the cells with Live/dead-Aqua dye (Invitrogen, Karlsruhe, Germany), CD8-FITC antibody clone SK1 (BD, Heidelberg, Germany) and PE- or APC-coupled A*2402 MHC multimers. For analysis, a BD LSRII SORP cytometer equipped with appropriate lasers and filters was used. Peptide specific cells were calculated as percentage of total CD8+ cells. Evaluation of multimeric analysis was done using the FlowJo software (Tree Star, Oreg., USA). In vitro priming of specific multimer+ CD8+ lymphocytes was detected by appropriate gating and by comparing to negative control stimulations. Immunogenicity for a given antigen was detected if at least one evaluable in vitro stimulated well of one healthy donor was found to contain a specific CD8+ T-cell line after in vitro stimulation (i.e. this well contained at least 1% of specific multimer+among CD8+ T-cells and the percentage of specific multimer+ cells was at least 10× the median of the negative control stimulations).

In Vitro Immunogenicity for IMA941 Peptides

For tested HLA class I peptides, in vitro immunogenicity could be demonstrated for three exemplary peptide by generation of peptide specific T-cell lines. Exemplary flow cytometry results after TUMAP-specific multimer staining for two peptides of the invention are shown in FIG. 3 together with a corresponding negative control. Results for one additional exemplary peptide from the invention are summarized in Table 4.

TABLE 4
Exemplary in vitro immunogenicity of HLA class
I peptides of the invention
The result of the in vitro immunogenicity
experiments conducted by the applicant are
showing the percentage of positive tested
donors and wells among evaluable for SEQ ID No 1.

| | | | Positive | Positive |
|---|---|---|---|---|
| | | | donors/ | wells/ |
| SEQ | | | donors tested | wells tested |
| ID NO: | Sequence | Allele | [%] | [%] |
| 1 | FYISPVNKL | A*24 | 100 | 50 |

### REFERENCE LIST

Abe F, Minegishi H (2008). Global screening of genes essential for growth in high-pressure and cold environments: searching for basic adaptive strategies using a yeast deletion library. Genetics 178, 851-872.
Allison J P, Krummel M F (1995). The Yin and Yang of T cell costimulation. Science 270, 932-933.
Aronov S, Gerst J E (2004). Involvement of the late secretory pathway in actin regulation and mRNA transport in yeast. J Biol. Chem. 279, 36962-36971.
Benjamini Y, Hochberg Y (1995). Controlling the False Discovery Rate: A Practical and Powerful Approach to Multiple Testing. Journal of the Royal Statistical Society. Series B (Methodological), Vol. 57, 289-300.
Bruckdorfer T, Marder O, Albericio F (2004). From production of peptides in milligram amounts for research to multi-tons quantities for drugs of the future. Curr. Pharm. Biotechnol. 5, 29-43.
Brunsvig P F, Aamdal S, Gjertsen M K, Kvalheim G, Markowski-Grimsrud C J, Sve I, Dyrhaug M, Trachsel S, Moller M, Eriksen J A, Gaudernack G (2006). Telomerase peptide vaccination: a phase I/II study in patients with non-small cell lung cancer. Cancer Immunol. Immunother. 55, 1553-1564.
Dengjel J, Nastke M D, Gouttefangeas C, Gitsioudis G, Schoor O, Altenberend F, Muller M, Kramer B, Missiou A, Sauter M, Hennenlotter J, Wernet D, Stenzl A, Rammensee H G, Klingel K, Stevanovic S (2006). Unexpected Abundance of HLA Class II Presented Peptides in Primary Renal Cell Carcinomas. Clin Cancer Res. 12, 4163-4170.
Dudley M E, Wunderlich J R, Robbins P F, Yang J C, Hwu P, Schwartzentruber D J, Topalian S L, Sherry R, Restifo N P, Hubicki A M, Robinson M R, Raffeld M, Duray P, Seipp C A, Rogers-Freezer L, Morton K E, Mavroukakis S A, White D E, Rosenberg S A (2002). Cancer regression and autoimmunity in patients after clonal repopulation with antitumor lymphocytes. Science 298, 850-854.
Dudley M E, Wunderlich J R, Yang J C, Sherry R M, Topalian S L, Restifo N P, Royal R E, Kammula U, White D E, Mavroukakis S A, Rogers L J, Gracia G J, Jones S A, Mangiameli D P, Pelletier M M, Gea-Banacloche J, Robinson M R, Berman D M, Filie A C, Abati A, Rosenberg S A (2005). Adoptive cell transfer therapy following non-myeloablative but lymphodepleting chemotherapy for the treatment of patients with refractory metastatic melanoma. J. Clin. Oncol. 23, 2346-2357.
Falk K, Rotzschke O, Stevanovic S, Jung G, Rammensee H G (1991). Allele-specific motifs revealed by sequencing of self-peptides eluted from MHC molecules. Nature 351, 290-296.
Gabrilovich D I, Chen H L, Girgis K R, Cunningham H T, Meny G M, Nadaf S, Kavanaugh D, Carbone D P (1996). Production of vascular endothelial growth factor by human tumors inhibits the functional maturation of dendritic cells. Nat. Med 2, 1096-1103.
Gattinoni L, Powell D J, Jr., Rosenberg S A, Restifo N P (2006). Adoptive immunotherapy for cancer: building on success. Nat. Rev. Immunol. 6, 383-393.
Giot L, Bader J S, Brouwer C, Chaudhuri A, Kuang B, Li Y, Hao Y L, Ooi C E, Godwin B, Vitols E, Vijayadamodar G, Pochart P, Machineni H, Welsh M, Kong Y, Zerhusen B, Malcolm R, Varrone Z, Collis A, Minto M, Burgess S, McDaniel L, Stimpson E, Spriggs F, Williams J, Neurath K, Ioime N, Agee M, Voss E, Furtak K, Renzulli R, Aanensen N, Carrolla S, Bickelhaupt E, Lazovatsky Y, DaSilva A, Zhong J, Stanyon C A, Finley R L, Jr., White K P, Braverman M, Jarvie T, Gold S, Leach M, Knight J, Shimkets R A, McKenna M P, Chant J, Rothberg J M (2003). A protein interaction map of Drosophila melanogaster. Science 302, 1727-1736.
Harsay E, Schekman R (2007). Avl9p, a member of a novel protein superfamily, functions in the late secretory pathway. Mol. Biol. Cell 18, 1203-1219.
Hill A B, Lee S P, Haurum J S, Murray N, Yao Q Y, Rowe M, Signoret N, Rickinson A B, McMichael A J (1995). Class I major histocompatibility complex-restricted cytotoxic T lymphocytes specific for Epstein-Barr virus (EBV)-transformed B lymphoblastoid cell lines against which they were raised. J Exp. Med 181, 2221-2228.
Ito T, Chiba T, Ozawa R, Yoshida M, Hattori M, Sakaki Y (2001). A comprehensive two-hybrid analysis to explore the yeast protein interactome. Proc Natl. Acad. Sci. U.S. A 98, 4569-4574.
Jerome K R, Domenech N, Finn O J (1993). Tumor-specific cytotoxic T cell clones from patients with breast and pancreatic adenocarcinoma recognize EBV-immortalized B cells transfected with polymorphic epithelial mucin complementary DNA. J Immunol 151, 1654-1662.
Jochmus I, Osen W, Altmann A, Buck G, Hofmann B, Schneider A, Gissmann L, Rammensee H G (1997). Specificity of human cytotoxic T lymphocytes induced by a human papillomavirus type 16 E7-derived peptide. J Gen. Virol. 78 (Pt 7), 1689-1695.
Jung G, Ledbetter J A, Muller-Eberhard H J (1987). Induction of cytotoxicity in resting human T lymphocytes bound to tumor cells by antibody heteroconjugates. Proc Natl Acad Sci USA 84, 4611-4615.
Kawakami T, Inagaki N, Takei M, Fukamachi H, Coggeshall K M, Ishizaka K, Ishizaka T (1992). Tyrosine phosphorylation is required for mast cell activation by Fc epsilon RI cross-linking J Immunol 148, 3513-3519.
Krieg A M (2006). Therapeutic potential of Toll-like receptor 9 activation. Nat. Rev. Drug Discov. 5, 471-484.
Ljunggren H G, Karre K (1985). Host resistance directed selectively against H-2-deficient lymphoma variants. Analysis of the mechanism. J Exp. Med 162, 1745-1759.
Longenecker B M, Reddish M, Koganty R, MacLean G D (1993). Immune responses of mice and human breast cancer patients following immunization with synthetic sialyl-Tn conjugated to KLH plus detox adjuvant. Ann N.Y. Acad. Sci. 690, 276-291.
Lu Gs, Mojsov S, Tam J P, Merrifield R B (1981). Improved synthesis of 4-alkoxybenzyl alcohol resin. The Journal of Organic Chemistry 46, 3433-3436.
Meziere C, Viguier M, Dumortier H, Lo-Man R, Leclerc C, Guillet J G, Briand J P, Muller S (1997). In vivo T helper cell response to retro-inverso peptidomimetics. J Immunol 159, 3230-3237.
Morgan R A, Dudley M E, Wunderlich J R, Hughes M S, Yang J C, Sherry R M, Royal R E, Topalian S L, Kammula U S, Restifo N P, Zheng Z, Nahvi A, de Vries C R, Rogers-Freezer L J, Mayroukakis S A, Rosenberg S A (2006). Cancer Regression in Patients After Transfer of Genetically Engineered Lymphocytes. Science.
Mueller L N, Rinner O, Schmidt A, Letarte S, Bodenmiller B, Brusniak M Y, Vitek O, Aebersold R, Muller M (2007). SuperHirn—a novel tool for high resolution LC-MS-based peptide/protein profiling. Proteomics. 7, 3470-3480.
Peoples G E, Goedegebuure P S, Smith R, Linehan D C, Yoshino I, Eberlein T J (1995). Breast and ovarian cancer-specific cytotoxic T lymphocytes recognize the same HER2/neu-derived peptide. Proc. Natl. Acad. Sci. U.S.A. 92, 432-436.
Pinheiro J, Bates D, DebRoy S, Sarkar D., R Core team. nlme: Linear and Nonlinear Mixed Effects Models. 2008.
Ref Type: Computer Program
Plebanski M, Allsopp C E, Aidoo M, Reyburn H, Hill A V (1995). Induction of peptide-specific primary cytotoxic T lymphocyte responses from human peripheral blood. Eur. J Immunol 25, 1783-1787.
Porta C, Spall V E, Loveland J, Johnson J E, Barker P J, Lomonossoff G P (1994). Development of cowpea mosaic virus as a high-yielding system for the presentation of foreign peptides. Virology 202, 949-955.
Rammensee H G, Bachmann J, Emmerich N P, Bachor O A, Stevanovic S (1999). SYFPEITHI: database for MHC ligands and peptide motifs. Immunogenetics 50, 213-219.
Rammensee, H G, Bachmann, J, Stevanovic, S (1997). MHC Ligands and Peptide Motifs. Springer-Verlag, Heidelberg, Germany).
Rini B I, Weinberg V, Fong L, Conry S, Hershberg R M, Small E J (2006). Combination immunotherapy with prostatic acid phosphatase pulsed antigen-presenting cells (provenge) plus bevacizumab in patients with serologic progression of prostate cancer after definitive local therapy. Cancer 107, 67-74.
Rosenberg S A, Lotze M T, Muul L M, Chang A E, Avis F P, Leitman S, Linehan W M, Robertson C N, Lee R E, Rubin J T. (1987). A progress report on the treatment of 157 patients with advanced cancer using lymphokine-activated killer cells and interleukin-2 or high-dose interleukin-2 alone. N. Engl. J. Med. 316, 889-897.
Rosenberg S A, Packard B S, Aebersold P M, Solomon D, Topalian S L, Toy S T, Simon P, Lotze M T, Yang J C, Seipp C A., (1988). Use of tumor-infiltrating lymphocytes and interleukin-2 in the immunotherapy of patients with metastatic melanoma. A preliminary report. N. Engl. J Med 319, 1676-1680.
Saiki R K, Gelfand D H, Stoffel S, Scharf S J, Higuchi R, Horn G T, Mullis K B, Erlich H A (1988). Primer-directed enzymatic amplification of DNA with a thermostable DNA polymerase. Science 239, 487-491.
Seeger F H, Schirle M, Gatfield J, Arnold D, Keilholz W, Nickolaus P, Rammensee H G, Stepvanovic S (1999). The HLA-A*6601 peptide motif: prediction by pocket structure and verification by peptide analysis. Immunogenetics 49, 571-576.
Small E J, Schellhammer P F, Higano C S, Redfern C H, Nemunaitis J J, Valone F H, Verjee S S, Jones L A, Hershberg R M (2006). Placebo-controlled phase III trial of immunologic therapy with sipuleucel-T (APC8015) in patients with metastatic, asymptomatic hormone refractory prostate cancer. J Clin Oncol. 24, 3089-3094.
Staehler M, Stenzl A, Dietrich P Y, Eisen T, Haferkamp A, Beck J, Mayer A, Walter S, Singh-Jasuja H, Stief C (Jun. 20, 2007). A phase I study to evaluate safety, immunogenicity and anti-tumor activity of the multi-peptide vaccine IMA901 in renal cell carcinoma patients (RCC). Journal of Clinical Oncology, 2007 ASCO Annual Meeting Proceedings Part I, Vol 25, No. 18S (June 20 Supplement), 2007: 5098.
Strubin M, Mach B, Long E O (1984). The complete sequence of the mRNA for the HLA-D R-associated invariant chain reveals a polypeptide with an unusual transmembrane polarity. EMBO J 3, 869-872.
Walter S, Herrgen L, Schoor O, Jung G, Wernet D, Buhring H J, Rammensee H G, Stevanovic S (2003). Cutting edge: predetermined avidity of human CD8 T cells expanded on calibrated MHC/anti-CD28-coated microspheres. J. Immunol. 171, 4974-4978.
Yee C, Thompson J A, Byrd D, Riddell S R, Roche P, Celis E, Greenberg P D (2002). Adoptive T cell therapy using antigen-specific CD8+ T cell clones for the treatment of patients with metastatic melanoma: in vivo persistence, migration, and antitumor effect of transferred T cells. Proc. Natl. Acad. Sci. U.S. A 99, 16168-16173.
Zhang L, Huang M, Harsay E (2010). A chemical genetic screen for modulators of exocytic transport identifies inhibitors of a transport mechanism linked to GTR2 function. Eukaryot. Cell 9, 116-126.

1. An AVL9 polypeptide having at least 85% homology to SEQ ID NO: 7.

2. The isolated peptide of claim 1 for use in medicine.

3. An isolated peptide comprising at least one sequence having at least 85% homology selected from the group consisting of SEQ ID NO: 1 to SEQ ID NO: 5 capable of inducing mammalian T cells cross-reacting with said peptide, wherein said peptide is not the full-length peptide according to SEQ ID NO: 7.

4. The peptide according to claim 3, wherein said peptide has the ability to bind to a molecule of the human major histocompatibility complex (MHC) class-I or -II.

5. The peptide according to claim 3 comprising a sequence selected from the group consisting of SEQ ID NO: 1 to SEQ ID NO: 5.

6. The peptide according to claim 3, wherein said peptide has the ability to bind to a molecule of the human major histocompatibility complex (MHC) class-I or -II.

7. The peptide according to claim 3, wherein said peptide is capable of stimulating CD4 or CD8 T cells.

8. The peptide according to claim 3, wherein said peptide has an overall length of not more than 100 amino acids.

9. The peptide according to claim 3, wherein said peptide has an overall length of not more than 30 amino acids.

10. The peptide according to claim 3, wherein said peptide has an overall length of not more than 16 amino acids.

11. The peptide according to claim 3, wherein said peptide has an overall length of not more than consisting of an amino acid sequence selected from the group consisting of SEQ ID NO: 1 to SEQ ID NO: 5.

12. The peptide according to claim 3 comprising chemically modified amino acids and/or non-peptide bonds.

13. The peptide according to claim 3, wherein said peptide is part of a fusion protein comprising N-terminal amino acids of the HLA-DR antigen-associated invariant chain (Ii).

14. A nucleic acid, encoding a peptide according to claim 3.

15. An expression vector capable of expressing the nucleic acid of claim 14.

16. A pharmaceutical composition comprising suitable pharmaceutical auxiliary agents and at least one entity selected from the group consisting of:
a. an AVL9 polypeptide having at least 85% homology to SEQ ID NO: 7
b. an isolated peptide according to claim 3,
c. a nucleic acid encoding the isolated peptide; and
d. an expression vector capable of expressing the nucleic acid.

17. The pharmaceutical composition according to claim 16, wherein said composition is an anti-cancer vaccine and optionally comprises at least one additional peptide comprising a sequence selected from the group consisting of any of SEQ ID 8 to 47.

18. An antibody capable of specifically binding to a peptide according to claim 3.

19. An activated cytotoxic T lymphocyte (CTL) which selectively recognizes a cell which aberrantly expresses a polypeptide comprising an amino acid sequence according to any of claim 3.

20. A host cell comprising an entity selected from the group consisting of:
a. recombinant nucleic acid according to claim 14, and
b. an expression vector capable of expressing the recombinant nucleic acid.

21. The host cell of claim 20, wherein the host cell is selected from the group consisting of an antigen presenting cell and a dendritic cell.

22. A method of treating a proliferative disease comprising administering to a subject in need thereof, an entity selected from the group consisting of:
a. an AVL9 polypeptide having at least 85% homology to SEQ ID NO: 7;
b. an isolated peptide according to claim 3,
c. a nucleic acid encoding the isolated peptide; and
d. an expression vector capable of expressing the nucleic acid; and
e. a host cell comprising said isolated peptide, nucleic acid, and/or expression vector.

23. The method of claim 22 wherein said proliferative disease is selected from the group consisting of cancer, gastric cancer, NSCLC, renal cell carcinoma, Benign prostatic hyperplasia, and colorectal carcinoma.

24. A method for diagnosing cancer, comprising detecting the presence of at least one peptide derived from the protein AVL9 presented on the surface of a cell and/or the level of expression of the gene AVL9 in a biological sample obtained from a mammal, wherein the presence of said peptide or an increase of the level of expression of the gene AVL9 in said sample compared to a biological non-cancer sample is indicative for cancer.

25. The method according to claim 24, wherein said cancer is selected from benign prostatic hyperplasia, gastric cancer, NSCLC, renal cell carcinoma, glioblastoma or colorectal carcinoma.

26. The method according to claim 24, wherein said detecting comprises contacting a sample with:
a. an antibody which specifically recognizes the AVL9 polypeptide,
b. an antibody capable of specifically binding to a peptide according to claim 3,
c. a fusion peptide comprising an AVL9-derived sequence, or
d. a nucleic acid capable of hybridizing under stringent conditions to a nucleic acid comprising SEQ ID NO: 6.

27. A diagnostic kit comprising:
a) a container containing a pharmaceutical composition according to claim 16 in solution or in lyophilized form;
b) optionally, a second container containing a diluent or reconstituting solution for the lyophilized formulation;
c) optionally, at least one peptide selected from the group consisting of the peptides according to SEQ ID NO: 8 to SEQ ID NO: 47;
d) optionally, primary and secondary antibodies, and suitable detection reagents, such as detectable moieties, enzyme substrates, and color reagents; and
e) optionally, instructions for (i) use of the solution or (ii) reconstitution and/or use of the lyophilized formulation.

28. The kit according to claim 27, further comprising one or more of (iii) a buffer, (iv) a diluent, (v) a filter, (vi) a needle, or (v) a syringe.

29. The kit according to claim 27, comprising components for detecting expression levels of AVL9 as a gastric cancer marker gene, said components selected from the group consisting of:
a) a control antibody which specifically binds to a gastric marker polypeptide,
b) one or more nucleic acids which capable of hybridizing to AVL9 mRNA under stringent conditions, and, optionally,
c) a control.

30. A method of producing an isolated peptide comprising at least one sequence having at least 85% homology selected from the group consisting of SEQ ID NO: 1 to SEQ ID NO: 5 capable of inducing mammalian T cells cross-reacting with said peptide, wherein said peptide is not the full-length peptide according to SEQ ID NO: 7, the method comprising:
a. culturing the host cell according to claim 20,
b. expressing the nucleic acid or the expression vector, and
c. isolating the peptide from said host cell or its culture medium.

31. A method for producing activated cytotoxic T lymphocytes (CTL) and/or T helper cells, wherein the method comprises contacting CTL in vitro with antigen loaded human class I or II MHC molecules expressed on the surface of a suitable antigen-presenting cell or an artificial construct mimicking an antigen-presenting cell for a period of time sufficient to activate said CTL in an antigen specific manner, wherein said antigen is a peptide according to any one of claim 3.

32. A method for killing target cells in a patient which target cells aberrantly express a polypeptide comprising an amino acid sequence selected from the group consisting of SEQ ID NO: 1 to SEQ ID NO: 5, wherein the method comprises administering to said patient an effective amount of cytotoxic T lymphocytes (CTL) as produced according to claim 31.

33. A method for treating or monitoring cancer in a patient, comprising a method for diagnosis according to claim 16, and treating said cancer in said patient based on said diagnostic result.
